(19) United States
(12) Patent Application Publication
(10) Pub. No.: US 2023/0078488 A1
(43) Pub. Date: Mar. 16, 2023

(54) METABOLITE FINGERPRINTING

(71) Applicant: Zymergen Inc., Emeryville, CA (US)

(72) Inventors: Erik Jedediah DEAN, Lafayette, CA (US); Stephen LOK, Alameda, CA (US); Ana Belen IBANEZ ZAMORA, El Cerrito, CA (US); Ee-Been GOH, Orinda, CA (US); Stefan BAUER, El Cerrito, CA (US); Franklin LU, Novato, CA (US)

(21) Appl. No.: 17/988,043

(22) Filed: Nov. 16, 2022

Related U.S. Application Data
(63) Continuation of application No. PCT/US2021/036439, filed on Jun. 8, 2021.
(60) Provisional application No. 63/036,637, filed on Jun. 9, 2020.

(51) Int. Cl. G16B 5/00; G16B 20/00; G16B 40/20

(52) U.S. Cl. CPC G16B 5/00; G16B 20/00; G16B 40/20

(57) ABSTRACT

The present disclosure provides methods for predicting phenotypic performance of a host cell in industrial culture. Specifically, the present disclosure provides methods for predicting phenotypic performance of a host cell in industrial culture by determining the metabolite fingerprinting profile of a host cell in small lab-scale culture and applying said profile to a predictive model of phenotypic performance.

### CROSS-REFERENCE TO RELATED APPLICATIONS

This application is a continuation of International Patent Application No. PCT/US2021/036439, filed Jun. 8, 2021, which claims priority to and the benefit of U.S. Provisional Patent Application No. 63/036,637, filed on Jun. 9, 2020, which is hereby incorporated by reference in its entirety including all descriptions, references, figures, and claims for all purposes.

### FIELD OF THE DISCLOSURE

The present disclosure generally relates to methods for predicting phenotypic performance of a host cell. Specifically, the present disclosure provides methods for predicting phenotypic performance of a host cell in industrial culture based on the metabolite profile of a host cell in small lab-scale culture.

### BACKGROUND

Microorganisms can be used to produce many industrial products. Selecting the microbial candidate most fit for production of the industrial product at a commercially viable scale is a cumbersome and time-consuming process which involves analyzing a large number of microbes over one or more generations.

One step in the process that is particularly inefficient is determining which microbes should be carried forward into large-scale development. Current high-throughput approaches identify lead microbes based on product measurements obtained in small lab-scale lab culture, which often do not translate to high product measurements during commercial development. Methods to accurately predict the phenotypic performance of microbes at a commercially viable scale would reduce costs and greatly speed up the production of high fitness microbes.

There remains an unmet need in the art for methods and tools to predict performance of microorganisms at a commercial scale.

### BRIEF SUMMARY

Embodiments of the present disclosure provide methods for predicting phenotypic performance of a host cell in industrial culture.

In some embodiments, the present disclosure provides a method for predicting phenotypic performance of a host cell based on chemical spectra of spent media, said method comprising the steps of: a) culturing a first host cell in nutrient media and separating the cultured cells from the media, thereby creating a first spent media; b) analyzing the first spent media via mass spectroscopy to generate a chemical spectra of said first spent media; c) providing a predictive model of phenotypic performance, said model comprising a metabolite fingerprint variable, and a phenotypic performance variable: i) wherein the metabolite fingerprint variable is based on chemical spectra of a plurality of spent media, each spent media having been derived from a host cell culture exhibiting a known phenotypic performance measurement; and ii) wherein the phenotypic performance variable is based on the known phenotypic performance measurement associated with each of the chemical spectra of part (i); and d) utilizing the predictive model to predict the expected phenotypic performance of the first host cell by providing the chemical spectra of the first spent media to the model.

In some embodiments, the present disclosure provides a computer-implemented method for predicting phenotypic performance of a host cell, said method comprising: a) accessing a training data set comprising a metabolite fingerprint variable, and a phenotypic performance variable; i) wherein the metabolite fingerprint variable comprises chemical spectra of a plurality of spent media, each spent media having been derived from a host cell culture exhibiting a known phenotypic performance measurement; and ii) wherein the phenotypic performance variable comprises the known phenotypic performance measurements that are associated with each of the spent media of part (i); b) developing a predictive model that is populated with the training data set; and c) utilizing the predictive model to predict the phenotypic performance of a first host cell by providing chemical spectra of a first spent media obtained from a culture of the first host cell to the predictive model; wherein the chemical spectra of spent media is measured via mass spectroscopy.

In some embodiments, the present disclosure provides a method for preparing a predictive model capable of predicting phenotypic performance of a host cell based on chemical spectra of spent media, said method comprising the steps of: a) providing a plurality of chemical spectra derived from mass spectroscopy analysis of spent media, each spent media having been derived from a lab-scale culture of a host cell exhibiting a known phenotypic performance measurement of the same host cell in an industrial-scale culture; and b) conducting a partial least squares analysis of the plurality of the chemical spectra of step (a) and their associated known phenotypic performance measurements, thereby generating a predictive model capable of predicting a first host cell's phenotypic performance in industrial scale cultures based on the chemical spectra of spent media. from a. lab-scale culture of the first host cell.

In some embodiments, the present disclosure provides a method for generating a metabolite fingerprint, said method comprising the steps of: a) obtaining a spent nutrient media sample from host cells in small-culture; b) analyzing the spent nutrient media sample by mass spectrometry; and c) processing the mass spectrometry data by spectral filtering, mass detection, chromatogram building, and peak alignment.

In some embodiments, the methods of the present disclosure provides a predictive model based on partial least squares regression of the chemical spectra of spent media and their associated known phenotypic performance measurements.

In some embodiments, the plurality of spent media in the metabolite fingerprint variable comprises at least 5, 10, 25, 50, 75, 100, 150, 200, or 250 chemical spectra.

In some embodiments, the metabolite fingerprint variable and phenotypic performance variable comprise the chemical spectra and the known phenotypic performance measurements from spent media from host cell cultures that exhibited a range of phenotypic performance measurements.

In some embodiments, the range of phenotypic performance measurements comprises at least a 2, 3, 4, 5, 6, 7, 8, 9, or 10-fold difference between the lowest and highest known phenotypic performance measurements.

In some embodiments, the range of phenotypic performance measurements comprises at least a 10%, 20%, 30%, 40%, 50%, 60%, 70%, 80%, or 90% difference between the lowest and highest known phenotypic performance measurements.

In some embodiments, ranges of phenotypic performance measurements for the metabolite fingerprint technologies of the present disclosure refer to relative changes of the lowest performing to the highest performing measurement. For example if a metabolic fingerprint variable comprises data for strains performing at 50% yield to 60% yield, then the “relative difference” between the lowest and highest measurements is 20%, which is calculated by the following formula: (highest measurement—lowest measurement)/lowest measurement.

In some embodiments, ranges of phenotypic performance measurements for the metabolite fingerprint technologies of the present disclosure refer to absolute changes in a phenotypic measurement. For example if a metabolic fingerprint variable comprises data for strains performing at 50% yield to 60% yield, then the “absolute difference” between the lowest and highest measurements is 10%, which is calculated by the following formula: (highest measurement—lowest measurement).

In some embodiments, the metabolite fingerprint variable comprises a chemical spectra for an anchor strain.

In some embodiments, the predicted phenotypic performance is production of a product of interest, said product of interest selected from the group consisting of: a small molecule, enzyme, protein, peptide, amino acid, organic acid, synthetic compound, fuel, alcohol, primary extracellular metabolite, secondary extracellular metabolite, intracellular component molecule, and combinations thereof.

In some embodiments, the model predicts the phenotypic performance of the first host cell in an industrial culture based on the chemical spectra of the first spent media obtained from a small lab-scale culture.

In some embodiments, the metabolite fingerprint variable is based on the chemical spectra of a plurality of spent media from small lab-scale cultures, and wherein the phenotypic performance variable is based on the known phenotypic performance measurements of the host cells in industrial cultures.

In some embodiments, the industrial culture is at least a 0.25, 0.5 1, 3, 4, 5, 6, 7, 8, 9, 10, 20, 30, 40, or 50 liter culture, and wherein the small lab-scale culture is less than about 5 mL, 4 mL, 3 mL, 2 mL culture, including all ranges and subranges therebetween. In some embodiments the small lab-scale culture is less than a 1000, 750, 500. 250, 200, 150, 100, 50, or 20 microliter culture.

In some embodiments, the small lab-scale culture is a culture from a single well in a 96-well plate.

As used herein the terms “small culture,” “small-scale culture,” “small scale culture,” “small lab culture,” “lab-scale culture” and similar are considered equivalents. Bench-scale cultures are considered industrial cultures for the purposes of this disclosure.

In some embodiments, the mass spectroscopy is direct injection electrospray ionization mass spectrometry. In some embodiments, the mass spectroscopy uses a time-of-flight spectrometer or analyzer.

In some embodiments, the chemical spectra are based on positive ion mass spectroscopy. in some embodiments, the chemical spectra are based on negative ion mass spectroscopy.

### BRIEF DESCRIPTION OF THE DRAWINGS

FIG. 1 shows a schematic of the workflow for predicting phenotypic performance of host cells in industrial culture.

FIG. 2 shows a schematic of the metabolite fingerprinting workflow for predicting phenotypic performance of host cells in industrial culture.

FIG. 3A shows a scatter plot of actual versus predicted performance in bench-scale fermenters. Predicted performance is based on metabolite fingerprinting of 5 or 10 distinct ladder strains and a reference (base) strain using partial least squares regression modeling.

FIG. 3B shows a scatter plot of actual versus predicted performance in bench-scale fermenters. Predicted performance is based on metabolite fingerprinting of 5 or 10 distinct ladder strains without a reference (base) strain using partial least squares regression modeling.

FIG. 4A shows a scatter plot of actual versus predicted performance in bench-scale fermenters. Predicted performance is based on metabolite fingerprinting of 5 distinct or clustered ladder strains and a reference (base) strain using partial least squares regression modeling.

FIG. 4B shows a scatter plot of actual versus predicted performance in bench-scale fermenters. Predicted performance is based on metabolite fingerprinting of 5 distinct or clustered ladder strains without a reference (base) strain using partial least squares regression modeling.

FIG. 5 shows a scatter plot of actual versus predicted performance in bench-scale fermenters. Predicted performance is based on 5 distinct ladder strains and a reference (base) strain in a plate-based assay (left panel) or metabolite fingerprinting (right panel).

FIG. 6A shows prediction of strain performance in bench-scale fermenters. Predicted performance is based on performance of 5 distinct ladder strains and a reference (base) strain in a plate-based assay. Each circle or diamond shape on the graph represents a particular strain.

FIG. 6B shows prediction of strain performance in bench-scale fermenters. Predicted performance is based on metabolite fingerprinting of 5 distinct ladder strains using partial least squares regression modeling. Each circle or diamond shape on the graph represents a particular strain.

FIG. 7 shows the accuracy of predicting phenotypic performance using a plate-based assay (left panel)) or metabolite fingerprinting (right panel),

FIG. 8 shows the probability distribution performance of the promoted strain (002) versus the reference strain (001) in bench-scale fermenters,

FIG. 9 illustrates other applications of metabolite profiling. The metabolite profile in plates versus tanks could be used to determine how well small lab-scale culture mimics industrial culture.

### DETAILED DESCRIPTION

The present disclosure provides methods for predicting phenotypic performance of a host cell in industrial culture.

### Definitions

As used herein the terms “host cell” “cellular organism”, “microorganism”, or “microbe” should be taken broadly. These terms are used interchangeably and include, but are not limited to, the two prokaryotic domains, Bacteria and Archaea, as well as certain eukaryotic fungi and protists.

The term “prokaryotes” is art recognized and refers to cells which contain no nucleus or other cell organelles. The prokaryotes are generally classified in one of two domains, the Bacteria and the Archaea. The definitive difference between organisms of the Archaea and Bacteria domains is based on fundamental differences in the nucleotide base sequence in the 16S ribosomal RNA.

The term “archaea” refers to a categorization of organisms of the division Mendosicutes, typically^{,} found in unusual environments and distinguished from the rest of the prokaryotes by several criteria, including the number of ribosomal proteins and the lack of muramic acid in cell walls. On the basis of ssrRNA analysis, the Archaea consist of two phylogenetically-distinct groups: Crenarchaeota and Euryarchaeota. On the basis of their physiology, the Archaea can be organized into three types: methanogens (prokaryotes that produce methane); extreme halophiles (prokaryotes that live at very high concentrations of salt (NaCl); and extreme (hyper) thermophiles (prokaryotes that live at very high temperatures). Besides the unifying archaeal features that distinguish them from Bacteria (1,e., no murein in cell wall, ester-linked membrane lipids, etc.), these prokaryotes exhibit unique structural or biochemical attributes which adapt them to their particular habitats. The Crenarchaeota consists mainly of hyperthermophilic sulfur-dependent prokaryotes and the Euryarchaeota contains the methanogens and extreme halophiles.

“Bacteria” or “eubacteria” refers to a domain of prokaryotic organisms. Bacteria include at least 11 distinct groups as follows: (1) Gram-positive (gram+) bacteria, of which there are two major subdivisions: (1) high G+C group (Actinomycetes, Mycobacteria, Micrococcus, others) (2) low G+C group (Bacillus, Clostridia, Lactobacillus, Staphylococci, Streptococci, Mycoplasmas); (2) Proteobacteria, e.g., Purple photosynthetic and non-photosynthetic Gram-negative bacteria (includes most “common” Gram-negative bacteria); (3) Cyanobacteria, e.g., oxygenic phototrophs; (4) Spirochetes and related species; (5) Planctomyces; (6) Bacteroides, Flavobacteria; (7)Chlamydia; (8) Green sulfur bacteria; (9) Green non-sulfur bacteria (also anaerobic phototrophs); (10) Radioresistant micrococci and relatives; (11) Therinoioga and Thermosipho thermophiles.

A “eukaryote” is any organism whose cells contain a nucleus and other organelles enclosed within membranes. Eukaryotes belong to the taxon Eukarya or Eukaryota. The defining feature that sets eukaryotic cells apart from prokaryotic cells (the aforementioned Bacteria and Archaea) is that they have membrane-bound organelles, especially the nucleus, which contains the genetic material, and is enclosed. by the nuclear envelope.

The term “titer” is defined as the strength of a solution or the concentration of a substance in solution. For example, the titer of a product of interest (e.g. small molecule, peptide, synthetic compound, fuel, alcohol, etc.) in a fermentation broth is described as g of product of interest in solution per liter of fermentation broth (g/L).

The term “culture” or “cell culture” means the maintenance of cells in an artificial, in vitro environment. “Nutrient medium” is used herein to refer to a nutrient solution for the culturing, growth, or proliferation of cells. Nutrient medium may be characterized by functional properties such as, but not limited to, the ability to maintain cells in a particular state (e.g., a proliferative state), or in some embodiments, the ability to produce a particular product.

As described above, the term “small lab-scale culture” refers to culture of a host cell in a nutrient media volume of less than about 5 mL, 4 mL, 3 mL, 2 mL, or 1 mL. In some embodiments, small lab-scale culture conditions mimic the conditions of industrial cultures.

The term “industrial culture” refers to culture of a host cell in a nutrient media volume of at least 0.25 L, 0.5 L, 0.6 L, 0.7 L, 0.8 L, 0.9 L, 1 L, 2 L, 3 L, 4 L, 5 L, 6 L, 7 L, 8 L, 9 L, 10 L, 11 L, 12 L, 13 L, 14 L, 15 L, 16 L, 17 L, 18 L, 19 L, 20 L, 30 L, 40 L, 50 L, 100 L, 200 L, 300 L, 400 L, 500 L, 750 L, 1,000 L, 5,000 L, 10,000 L, 25,000 L, 50,000 L, 75,000 L or 100,000 L, including all ranges and subranges therebetween. In some embodiments, a small lab-scale culture (i.e., a nutrient media volume ≤5 mL or 0.005 L) accurately mimics phenotypic performance in an industrial culture (i.e., a nutrient media. volume ≥0.25 L). In some embodiments, an industrial culture is a bench-scale culture (i.e., a culture with a nutrient media volume ranging from about 0.25 L to about 20 L). In some embodiments, an industrial culture is a production-scale culture (i.e., a culture with a nutrient media volume greater than 20 L). industrial culture of microorganisms is described by Cipriano. 2006. Large-Scale Production of Microorganisms, p 561-577. In Fleming D, Hunt D (ed), Biological Safety and Hans-Peter Meyer et al., Industrial-Scale Fermentation, Industrial Biotechnology: Products and Processes, 2017, herein incorporated by reference in their entirety.

In some embodiments, the industrial culture is a bench-scale industrial culture (e.g., bench-top bioreactors, or fermenters). In some embodiments, the bench-scale industrial culture is at least 0.25 L, 0.5 L, 0.6 L, 0.7 L, 0.8 L, 0.9 L, 1 L, 2 L, 3 L, 4 L, 5 L, 6 L, 7 L, 8 L, 9 L, 10 L, 11 L, 12 L, 13 L, 14 L, 15 L, 16 L, 17 L, 18 L, 19 L, or 20 L, including all ranges and subranges therebetween. In some embodiments, a small lab-scale culture accurately mimics phenotypic performance in a bench-scale industrial culture. In some embodiments, the methods described herein are used to determine phenotypic performance in bench-scale industrial culture. In some embodiments, the host cells of the present disclosure are bench-scale cultured in 0.25 L nutrient medium.

In some embodiments, the industrial culture is production-scale industrial culture (i.e., a culture with a nutrient media volume greater than 20 L). In some embodiments, the industrial culture is at least 30 L, 40 L, 50 L, 100 L, 200 L, 300 L, 400 L, 500 L, 750 L, 1,000 L, 5,000 L, 10,000 L, 25,000 L, 50,000 L, 75,000 L or 100,000 L, including all ranges and subranges therebetween. In some embodiments, a small lab-scale culture accurately mimics phenotypic performance in a production-scale industrial culture. In some embodiments, the methods described herein are used to determine phenotypic performance in a production-scale industrial culture.

The term “metabolite” refers to any substance within a host cell or produced by a host cell, including secreted substances, which can be quantitatively determined by applying analytical methods known in the art, e.g, methods that can resolve mass differences between metabolites.

The term “metabolite fingerprint” or “metabolite profile” refers to the presently disclosed methodology of analyzing spent nutrient media from small lab-scale culture to predict phenotypic performance in industrial culture. In some embodiments, “metabolite fingerprint” is also used in reference to a variable that comprises chemical spectra information in the training data sets of the present disclosure.

The term “training dataset” refers to a dataset for which a classification may be known. In some embodiments, the training dataset comprises information about a plurality of host cells, each with a known metabolite profile (i.e. metabolite fingerprint variable comprising a chemical spectra) and an associated phenotypic performance measurement (i.e., phenotypic performance variable). The training dataset is used to generate a transfer function for the prediction of phenotypic performance of an independent “unknown” host cell. The “training dataset” may also be referred to as the “ladder strain” or “strain ladder” throughout the disclosure.

As used herein, the term “reference strain” refers to a strain in the ladder set known to perform stably in industrial culture conditions. In some embodiments, the reference strain is a. top performer in industrial culture. In some embodiments, the reference strain is a mid-range performer in industrial culture. In some embodiments, the reference strain is a low-range performer in industrial culture. Performance can be measured by a variety of factors such as product yield or overall titer.

As used herein, the term “anchor strain” or “base strain” refers to a host strain exhibiting a basal level of performance, typically significantly lower than other engineered strains. Statistical functions built with “anchor” and/or “base” strains tend to exhibit an anchoring effect on the statistical model due to their low performance.

As used herein, “chromatography” refers to a process in which a chemical mixture carried by a liquid or gas is separated into components as a result of differential distribution of the chemical entities as they flow around or over a stationary liquid or solid phase.

As used herein, “liquid chromatography” (LC) means a process of selective retardation of one or more components of a fluid solution as the fluid uniformly percolates through a column of a finely divided substance, or through capillary passageways. The retardation results from the distribution of the components of the mixture between one or more stationary phases and the bulk fluid, (i.e., mobile phase), as this fluid moves relative to the stationary phase(s). “Liquid chromatography” includes reverse phase liquid chromatography (RPLC), high performance liquid chromatography (HPLC) and high turbulence liquid chromatography (HTLC).

As used herein, the term “HPLC” or “high performance liquid chromatography” refers to liquid chromatography in which the degree of separation is increased by forcing the mobile phase under pressure through a stationary phase, typically a densely packed column.

As used herein, the term “gas chromatography” refers to chromatography in which the sample mixture is vaporized and injected into a stream of carrier gas (as nitrogen or helium) moving through a column containing a stationary phase composed of a liquid or a particulate solid and is separated into its component compounds according to the affinity of the compounds for the stationary phase.

As used herein, “mass spectrometry” (MS) refers to an analytical technique to identify compounds by their mass. MS technology generally includes (1) ionizing the compounds to form charged compounds; and (2) detecting the molecular weight of the charged compound and calculating a mass-to-charge ratio (m/z). The compound may be ionized and detected by any suitable means. A “mass spectrometer” generally includes an ionizer and an ion detector. See, e.g., U.S. Pat. No. 6,204,500, entitled “Mass Spectrometry From Surfaces;” U.S. Pat. No. 6,107,623, entitled “Methods and Apparatus for Tandem Mass Spectrometry,” U.S. Pat. No. 6,268,144, entitled “DNA Diagnostics Based On Mass Spectrometry;” U.S. Pat. No. 6,124,137, entitled “Surface-Enhanced Photolabile Attachment And Release For Desorption And Detection Of Analytes;” Wright et al., Prostate Cancer and Prostatic Diseases 2:264-76 (1999); and Merchant and Weinberger, Electrophoresis 21:1164-67 (2000).

The term “time-of-flight mass spectrometry” refers to a method of mass spectrometry in which an ion's mass-to-charge ratio is determined via the time of flight measurement.

The term “electrospray ionization” or “ESI” refers to methods in which a solution is passed along a short length of capillary tube, to the end of which is applied a high positive or negative electric potential. Solution reaching the end of the tube is vaporized (nebulized) into a jet or spray of very small droplets of solution in solvent vapor. This mist of droplets flows through an evaporation chamber, which is heated slightly to prevent condensation and to evaporate solvent. As the droplets get smaller the electrical surface charge density increases until such time that the natural repulsion between like charges causes ions as well as neutral molecules to be released.

The term “ionization” as used herein refers to the process of generating an analyte ion having a net electrical charge equal to one or more electron units. Negative ions are those having a net negative charge due to the gain of one or more electron units, while positive ions are those having a net positive charge due to the loss of one or more electron units.

The term “operating in negative ion mode” refers to those mass spectrometry methods where negative ions are detected. Similarly, “operating in positive ion mode” refers to those mass spectrometry methods where positive ions are detected.

The term “overfitting” refers to when a statistical model describes random error or noise instead of the underlying relationship. Overfitting produce misleading coefficients, R-squared, and p-values, resulting in a model that fits the sampled data perfectly but that will fail to predict new data well.

The term “dimensionality” refers to the number of variables under consideration in a data. set. The term “noise” refers to the presence of any signal in the dataset other than the signals which are desired for analysis. As used herein in reference to mass spectrometry, the term “noise” means low abundant inconsistent chemical-based and electronics-based signal. The phrase “reduce the noise and dimensionality” refers to the use of signal filtering and statistical techniques to reduce the number of variables in the datasets and improve the signal-to-noise ratio of the data.

As used herein, unless otherwise stated, the singular forms “a,” “an,” and “the” include plural reference. Thus, for example, a reference to “a. protein” includes a plurality of protein molecules.

### Traditional Approaches for Predicting Phenotypic Performance

The cultivation of microorganisms in industrial cultures to produce a product is typically undertaken for a commercial purpose and a process developed in the laboratory must be translated into a process at full manufacturing scale.

The environment of industrial cultures is drastically different from that of small lab-scale cultures and it has long been recognized that most microorganisms do not perform the same way under both conditions (Humphrey A. Biotechnol Progress. 1998; 14: 3-7.) Scale-down models have been used for predicting phenotypic performance of host cells in industrial cultures. This approach generates and tests large numbers of microbial strains under conditions representative of large-scale growth and production, allowing for the selection of strains that will scale-up more predictably (Takors, R. Journal of Biotechnology, 2012; 160(1-2):3-9),

One traditional scale-down approach is the use of a plate-based assay to predict phenotypic performance. Strains in the plate-based assay are promoted to industrial cultures (e.g., a culture with a nutrient media volume ≥0.25 L) based on three criteria: optical density in seed plates, overall titers, and. titer/reactants consumed. Traditional plate promotion using this method is costly, time-consuming, and often inaccurate. As with other traditional scale-down approaches, it is difficult to identify strains that maintain the desired production characteristics when transitioned to industrial culture for the purposes of manufacturing.

The methods, systems and tools of the present disclosure address the shortcomings of the prior art by providing improved predictions of industrial-scale performance based on small lab-scale performance (e.g., ≤0.005 L or 5 mL), as measured by chemical spectra of spent nutrient media. The details of this new invention are described below.

### Methods, Systems, and Tools for Predicting Phenotypic Performance

The present disclosure provides methods, systems, and tools for predicting phenotypic performance of host cells in industrial culture.

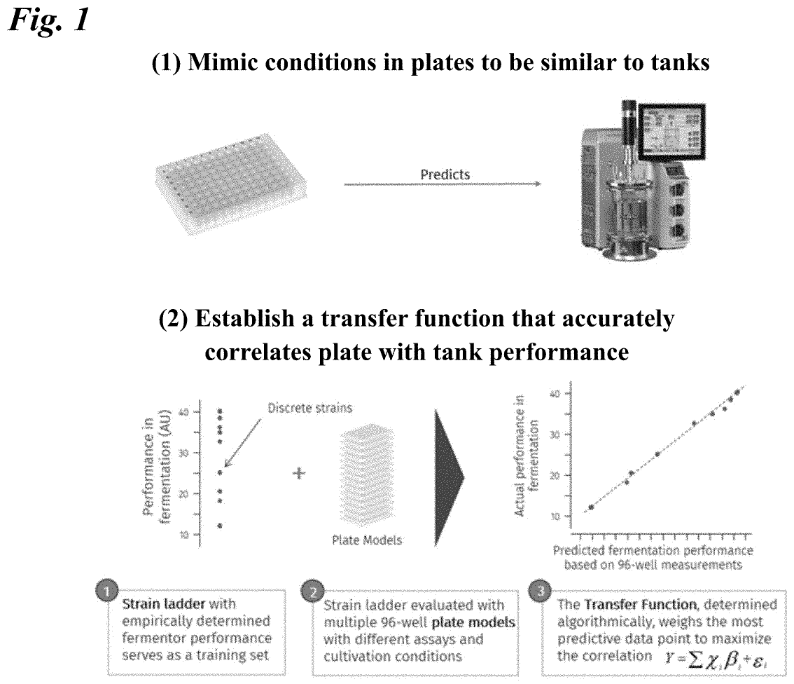

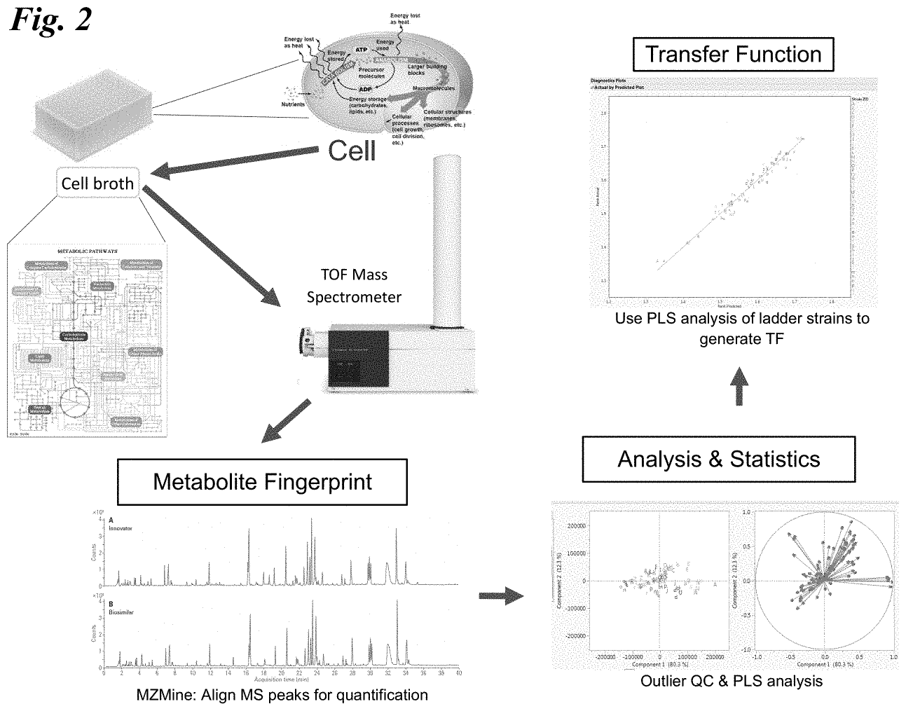

In some embodiments, the present disclosure teaches a workflow for predicting phenotypic performance of host cells comprising: 1) culturing host cells in nutrient media and separating the cultured cells from the nutrient media, thereby creating spent nutrient media, 2) analyzing the spent nutrient media by mass spectrometry to generate a chemical spectra of spent nutrient media, and 3) providing a predictive model of phenotypic performance based on a metabolite fingerprint variable (comprising data from the chemical spectra) and a phenotypic performance variable, and 4) utilizing the model to predict the phenotypic performance of the host cell by providing the chemical spectra of the spent nutrient media. The general workflow for predicting phenotypic performance is shown in FIG. 1 and FIG. 2 of the present disclosure.

Generating a Metabolite Fingerprint from Small Lab-Seale Culture Sample collection

The first step in predicting phenotypic performance is small lab-scale culture of host cells under aseptic conditions. The cultivation conditions vary significantly by host cell species and project specific strains. Host cells are seeded in multiwell plates in nutrient media and grown under fixed culture conditions (time, temperature, agitation). The nutrient media is then separated from the host cells, thereby creating spent nutrient media, which is used to predict phenotypic performance of the host cell. The separation of cells from nutrient media can be conducted by any known means in the art. In some embodiments, small lab-scale cultures are centrifuged or filtered to remove particulate matter (e.g., cells) from the cultures prior to analysis by mass spectrometry.

Host cells of the present disclosure can be cultured in conventional nutrient media modified as appropriate for any desired biosynthetic reactions or selections. In some embodiments, the present disclosure teaches using media that mimics the conditions of industrial cultures of the microorganism a culture with a nutrient media volume of 0.5 L or greater). In some embodiments, the culture conditions at industrial scale are provided, either by the client, or through a review of literature for the microorganism being used. Thus, in some embodiments, the present disclosure teaches nutrient media optimized for cell growth. In other embodiments, the present disclosure teaches nutrient media optimized for product yield. In some embodiments, the present disclosure teaches nutrient media capable of inducing cell growth and also contains the necessary precursors for final product production (e.g., high levels of sugars for ethanol production). In some embodiments, the present disclosure teaches nutrient media with selection agents, including selection agents of transformants (e.g., antibiotics), or selection of organisms suited to grow under inhibiting conditions (e.g., high ethanol conditions).

Culture conditions, such as temperature, pH and the like, are those suitable for use with the host cell selected for the methods of the present disclosure, and will be apparent to those skilled in the art. Many references are available for culturing host cells, including cells of bacterial, plant, animal (including mammalian) and archaeba.cterial origin. See, e.g., Sambrook, Ausubel (all supra), as well as Berger, Guide to Molecular Cloning Techniques, Methods in Enzymology volume 152 Academic Press, Inc., San Diego, Calif.; and. Freshney (1994) Culture of Animal Cells, a Manual of Basic Technique, third edition, Wiley-Liss, N.Y. and the references cited therein; Doyle and Griffiths (1997) Mammalian Cell Culture: Essential Techniques John Wiley and Sons, NY; Humason (1979) Animal Tissue Techniques, fourth edition W.H. Freeman and Company; and Ricciardelle et at, (1989) In Vitro Cell Dev. Biol.. 25:1016-1024; Life Science Research Cell Culture Catalogue from Sigma-Aldrich, Inc (St Louis, Mo.) (“Sigma-LSRCCC”), all of which are incorporated herein by reference.

The nutrient medium to be used must in a suitable manner satisfy the demands of the respective strains. Descriptions of culture media for various microorganisms are present in the “Manual of Methods for General Bacteriology” of the American Society for Bacteriology (Washington D.C., USA, 1981). Cell culture nutrient media in general is set forth in Atlas and Parks (eds.) The Handbook of Microbiological Media (1993) CRC Press, Boca Raton, Fla., which is incorporated herein by reference.

Host cells of the present disclosure may be cultured in flasks, tubes, dishes, or multiwell plates. Multi well plates include, but are not limited to, 6 well, 12 well, 24 well, 48 well, 96 well plates. The multiwell plates may be U-bottom, V-bottom, Flat (F)-bottom, or C-bottom shaped multiwell plates. In some embodiments, the host cells of the present disclosure are cultured in 96 well, F-bottom plates. In some embodiments, the host cells of the present disclosure are cultured in 96-well, U-bottom plates. In some embodiments, the host cells of the present disclosure are cultured in 96-well, V-bottom plates. In some embodiments, the small lab-scale culture used for predicting phenotypic performance of host cells is a culture from a single well in a 96-well plate.

Host cells can be cultured in nutrient medium at a. density that permits cell expansion and/or promotes production of a particular product (e.g., an amino acid, protein, or enzyme). Particular growth conditions may vary to accommodate various host cells and applications. Persons having skill in the art will be familiar with various culture conditions utilized to the desired industrial application of host cells. In some embodiments, the methods of the disclosure comprise culturing host cells at a density of at least 10² cells, 10³ cells, 10⁴ cells, 10⁵ cells, 10⁶ cells, 10⁷ cells, 10⁸ cells, 10⁹ cells, 10¹⁰ cells, 10¹¹ cells, 10¹² cells, 10¹³ cells, 10¹⁴ cells, 10¹⁵ cells per mL, or any number of cells there between. In some embodiments, the host cells are cultured at a density of 10⁸.

In some embodiments, the host cells can be incubated in nutrient medium for about 1 hours, 2 hours, 3 hours, 4 hours, 5 hours. 6 hours, 7 hours, 8 hours, 9 hours, 10 hours, 11 hours, 12 hours, 13 hours, 14 hours, 15 hours, 16 hours, 17 hours, 18 hours, 19 hours, 2( )hours, 21 hours, 22 hours, 23 hours, 24 hours, 25 hours, 26 hours. 27 hours, 28 hours, 29 hours, 30 hours, 31 hours, 32 hours, 33 hours, 34 hours, 35 hours, 36 hours, 37 hours, 38 hours, 39 hours, 40 hours, 41 hours, 42 hours, 43 hours, 44 hours, 45 hours, 46 hours, 47 hours, or 48 hours. In some embodiments, the host cells can be incubated in nutrient medium for about 1 day to about 30 days, or about 2 days to about 27 days, or about 3 days to about 25 days, or about 4 days to about 23 days, or about 5 days to about 20 days, or about 6 days to about 20 days, or about 7 days to about 15 days, or about 10 days to about 20 days, In some embodiments, host cells of the present disclosure are cultured in nutrient medium for 48 hours before subjecting the spent nutrient medium for downstream analysis of predicted phenotypic performance.

In some embodiments, the host cells are cultured on a small lab-scale. In some embodiments, small lab-scale culture is at less than 5 mL, 4 mL, 3 mL, 2 mL, 1 mL, 750 μL, 500 μL, 250 μL, 200 μL, 150 μL, 100 μL, 50 μL, 40 μL, or 20 μL, or 10 μL of nutrient media, in some embodiments, host cells of the present disclosure are cultured in 300 μL of nutrient media to generate spent nutrient media for mass spectrometry analysis and prediction of phenotypic performance.

### Mass Spectrometry

The next step in predicting phenotypic performance is subjecting spent nutrient media obtained from small lab-scale culture of host cells to mass spectrometry. In some embodiments, the detection step provides a compositional profile of the spent nutrient media. In some embodiments the mass spectrometry detects and quantifies the presence of one or more metabolites in the spent nutrient media. Analysis of metabolites by mass spectrometry is further described by Witt et al., Ultrafast Statistical Profiling of Bacterial Metabolite Extracts, Application Note FTMS-51, Bruker; Marques et al., Rapid Communications in Mass Spectrometry, 2006; 20:3654-3658; Liu et al., Rapid Communications in Mass Spectrometry, 2010; 24:1365-1370; herein incorporated by reference in their entirety.

### Sample Preparation:

In some embodiments, the spent nutrient media from small lab-scale cultures is processed and prepared prior to mass spectrometry analysis. Persons having skill in the art of mass spectroscopy will be familiar with the various methods for preparing samples for analysis.

In some embodiments, the present disclosure teaches methods of concentrating or diluting spent nutrient media prior to analysis. For example, in some embodiments, methods may be used prior to mass spectrometry to increase the concentration of particular metabolites in the spent nutrient medium sample. Such methods include, for example, filtration, centrifugation, thin layer chromatography (TLC), electrophoresis, affinity separation, extraction methods, and the use of chaotropic agents or any combination of the above or the like. Extraction methods include but are not limited to hexanes, dichloromethane, ethyl acetate, methanol, isopropanol, water, aqueous ethanol (ethanol:water, 70:30 v/v), and a dichloromethane/methanol mix (dichloromethane:methanol, 1:1 v/v), Extraction methods are further described by Martin et al., RSC Advances, 2014; 50; Zhang et al., Chinese Medicine, 2018; 20(13); Moldoveanu and David, Solvent Extraction, Modern Sample Preparation for Chromatography, 2015 pp. 131-189; herein incorporated by reference in their entirety.

In some embodiments, the spent nutrient media is diluted to ensure that the chemical concentrations of the spent nutrient media are within the dynamic range of the instrument. In some embodiments, the spent nutrient media is diluted to about 1:10, 1:100, 1:250, 1:500, 1:1,000, 1:5,000 or 1:10,000, including all ranges and subranges therebetween. In some embodiments, the spent nutrient medium is diluted to about 1:500. In other embodiments, the spent nutrient media is diluted to about 1:1,000.

Spent nutrient media may be further processed or purified to obtain preparations that are suitable for analysis by mass spectrometry. Such purifications may include chromatography, such as liquid chromatography, and may also often involve an additional purification procedure that is performed prior to chromatography. Various procedures may be used for this purpose depending on the type of sample or the type of chromatography. Other examples include filtration, extraction, precipitation, centrifugation, delipidization, desalting, dilution, combinations thereof and the like. In some embodiments, protein precipitation is used to remove most of the protein (when the product of interest is not a protein) from the sample leaving other components (e.g., other metabolites) soluble in the supernatant. The samples can be centrifuged to separate the liquid supernatant from the precipitated proteins. The resultant supernatant can then be applied to liquid chromatography and subsequent mass spectrometry analysis. Such protein purification methods are well known in the art, for example, Poison et al., Journal of Chromatography B 785:263-275 (2003), describes protein precipitation methods suitable for use in the methods of the disclosure. In some embodiments, desalting is used to remove salts from the spent nutrient media before the spent nutrient media is analyzed by mass spectrometry. Desalting methods are well known in the art, for example, Gundry et al., Curr Protoc Mol Biol, (2009), describes desalting methods suitable for use in the methods of the present disclosure.

In some embodiments, chromatography is performed prior to mass spectrometry. In some embodiments, the spent nutrient media sample is purified by liquid chromatography. In some embodiments, the spent nutrient media sample is purified by high performance liquid chromatography (HPLC). Various methods have been described involving the use of HPLC for sample clean-up prior to mass spectrometry analysis. See, e.g., Taylor et al., Therapeutic Drug Monitoring 22:608-12 (2000); and Salm et al., Clin. Therapeutics 22 Supl, B:B71-B85 (2000).

### Detection and Quantitation of Metabolites by Mass Spectrometry:

Methods of the present disclosure include detecting the presence of one or more metabolites in a spent nutrient media sample to predict phenotypic performance of a host cell. In some embodiments, the method comprises: 1) ionizing the metabolite(s), 2) detecting the ion(s) by mass spectrometry, and 3) relating the presence or amount of the ion(s) to the presence or amount of the metabolite(s) in the sample.

Mass spectrometry is performed using a mass spectrometer which includes an ion source for ionizing the spent nutrient media sample and creating charged molecules for further analysis. For example, ionization of the sample may be performed by electrospray ionization (ESI), atmospheric pressure chemical ionization (APCI), photoionization, electron ionization, fast atom bombardment (FAB)/liquid secondary ionization (LSIMS), matrix assisted laser desorption ionization (MALDI), field ionization, field desorption, thermospray/plasmaspray ionization, and particle beam ionization. The skilled artisan will understand that the choice of ionization method can be determined based on a number of factors, including but not limited to, the type of sample, the type of detector and the choice of positive versus negative mode.

After the sample has been ionized, the positively charged or negatively charged ions may be analyzed to determine a mass-to-charge ratio (i.e.. m/z). Suitable analyzers for determining mass-to-charge ratios include time-of-flight analyzers, quadrupole analyzers, and ion traps analyzers. The ions may be detected using several detection modes. For example, selected ions may be detected (i.e., using a selective ion monitoring mode (SIM)), or alternatively, ions may be detected using a scanning mode, e.g., full scan, multiple reaction monitoring (MRM), or selected reaction monitoring (SRM)). In some embodiments, the ions are detected using full scan mode. 100911 In some embodiments, the mass-to-charge ratio is determined using a time-of-flight analyzer. Ions are accelerated by an electric field of known strength which results in an ion having the same kinetic energy as any other ion that has the same charge. The velocity of the ion depends on the mass-to-charge ratio (e.g., heavier ions of the same charge reach lower speeds). The time that it takes for the ion to reach a detector at a known distance is measured. This time will depend on the velocity of the ion, and therefore is a measure of its mass-to-charge ratio. From this ratio and known experimental parameters, one can identify the ion.

One may enhance the resolution of the MS technique by employing “tandem mass spectrometry,” or “MS/MS.” In this technique, a precursor ion (also called a parent ion) generated from a molecule of interest can be filtered in an MS instrument, and the precursor ion is subsequently fragmented to yield one or more fragment ions (also called daughter ions or product ions) that are then analyzed in a second MS procedure. By careful selection of precursor ions, only ions produced by certain analytes are passed to the fragmentation chamber, where collision with atoms of an inert gas to produce the daughter ions. Because both the precursor and fragment ions are produced in a reproducible fashion under a given set of ionization/fragmentation conditions, the MS/MS technique can provide an extremely powerful analytical tool. For example, the combination of filtration/fragmentation can be used to eliminate interfering substances, and can be particularly useful in complex samples, such as biological samples.

Additionally, recent advances in technology, such as matrix-assisted laser desorption ionization coupled with time-of-flight analyzers (“MALDI-TOF”) permit the analysis of analytes at femtomole levels in very short ion pulses. Mass spectrometers that combine time-of-flight analyzers with tandem MS are also well known to the artisan. Additionally, multiple mass spectrometry steps can be combined in methods known as “MS/MSⁿ.” Various other combinations may be employed, such as MS/MS/TM MALDI/MS/MS/TOF, or SELDI/MS/MS/TOF mass spectrometry.

As ions collide with the detector, they produce a pulse of electrons that are converted to a digital signal. The acquired data is relayed to a computer, which plots counts of the ions collected versus time. The resulting mass chromatograms are similar to chromatograms generated in traditional HPLC methods. The areas under the peaks corresponding to particular ions, or the amplitude of such peaks, are measured and the area or amplitude is correlated to the amount of the analyte (e.g., metabolite) of interest. In some embodiments, the area under the curves, or amplitude of the peaks, for fragment ion(s) and/or precursor ions are measured to determine the amount of metabolite in the spent nutrient media sample.

In some embodiments, mass spectrometer measurements were performed in centroid mode. Centroid mode provides a compressed data file in which mass-to-charge peaks have no width, only peak intensities.

The methods of predicting phenotypic performance may involve mass spectrometry performed in either positive or negative ion mode. In some embodiments, the mass spectrometer operates in negative ion mode. In other embodiments, the mass spectrometer operates in positive mode.

In some embodiments, the methods of the present disclosure do not require identification of specific components in the spent nutrient media. That is, in some embodiments, the transfer function methods utilize the chemical spectra produced by the mass spectrometry analysis, without associating individual peaks with any particular molecule. Thus, in some embodiments, the methods of the present disclosure are agnostic to the particular components present in the spent nutrient media sample.

In some embodiments, the ion scan, i.e., mass spectrum, can be related to the amount of specific analytes in the spent nutrient media sample. For example, given that sampling and analysis parameters are carefully controlled, the relative abundance of a given ion can be compared to a table that converts that relative abundance to an absolute amount of the original molecule. Alternatively, molecular standards can be run with the samples, and a standard curve constructed based on ions generated from those standards. Using such a standard curve, the relative abundance of a given ion can be converted into an absolute amount of the original molecule. Numerous other methods for relating the presence or amount of an ion to the presence or amount of the original molecule are well known to those of ordinary skill in the art.

Several atmospheric ionization mass spectrometry (MS) techniques may be utilized for metabolite analysis of the spent nutrient medium, including but not limited to, time of flight mass spectrometry (TOF-MS), desorption electrospray ionization mass spectrometry (DESI-MS), extractive electrospray ionization mass spectrometry (EESI-MS), gas chromatography coupled mass-spectrometry (GC-MS), and direct analysis in real time (DART).

### Processing and Analysis of Mass Spectral Data:

The present disclosure utilizes MS analytical software to process mass spectrometry data. The analytical software described in the present disclosure processes the mass spectral data by performing: i) spectral filtering, ii) mass detection, iii) chromatogram building, and iv) peak alignment. The processed chemical spectra are then used to generate a predictive model of phenotypic performance (from e.g., one or more ladder strains) or the processed chemical spectra data (from e.g., a test strain or independent host cell) is provided to a model in order to predict phenotypic performance of a host cell in industrial culture.

Persons having skill in the art will be familiar with methods of processing and analyzing mass spectrometry data. Methods of analyzing mass spectrometry data are further described in Antoniadis et al., J Soc Fr Stat, 2010; 151, incorporated by reference in its entirety. For the sake of completeness, a brief disclosure of processing/analysis steps envisioned by the present disclosure are provided below. The descriptions below use an mzMine software to illustrate the process. Persons having skill in the art will appreciate that the same steps may be carried out through a variety of other software packages.

In some embodiments, the mass spectrometry data generated from the spent nutrient media undergoes data processing. As used herein, the term “data processing” refers to statistical analyses of the raw data in order to reduce the noise and dimensionality of the data, as well as reduce potential weighting of the data towards specific metabolites that may produce many mass-to-charge signals. In some embodiments, the data generated by the mass spectrometer are processed by performing spectral filtering, mass detection, chromatogram building, and peak alignment.

In some embodiments, the data files obtained from analyzing the spent nutrient media sample by mass spectrometry are converted to the open-source file type .mzML before further processing is performed, as described below. In some embodiments, the data files are converted to the open-source .mzML file type using the msconvert application in ProteoWizard. A brief description of ProteoWizard is provided in Table 1 below.

### i) Spectral Filtering

In some embodiments, the mass spectra data undergo spectral filtering. The term “spectral filtering” refers to the removal of random noise, typically of electronic or chemical origin. In some embodiments, spectral filtering was performed to remove candidate peaks with a signal intensity below a certain threshold. In some embodiments, the mass spectra data is filtered to remove samples that had poor injections. A poor injection would be indicated by the absence of peaks or variable peak sizes with expected peaks being too high or too low.

### ii) Mass Detection

In some embodiments, mass detection (also referred to as peak detection) is then performed to find. the peaks in the measurement data. Peak picking methods include, but are not limited to, the local maximum method and recursive threshold method. The local maximum method treats every local intensity maximum along the spectrum as a spectral peak, while the recursive threshold method requires the maximum to have a user-definable width that differentiates it from sharper noise peaks. Choice of methods for peak detection depends on the nature of input data and can easily be determined by one of skill in the art. In some embodiments, the parameters for mass detection comprise a scan range of 1-161, a retention time range of 0.05-0.15 minutes, MS level of 1, a centroided spectrum type, and a noise level of either 500 or 1000. In some embodiments, the parameters for mass detection are set to negative polarity. In other embodiments, the parameters for mass detection are set to positive polarity.

iii) Chromatogram Building

The next step in mass spectra data processing is chromatogram building. A chromatogram is constructed for each of the mass values which span over a certain time range. The number of peaks vary by the nature of the sample and other factors readily identified by those of skill in the art. In some embodiments, deconvolution algorithms are applied to each chromatogram to recognize the actual chromatographic peaks. In some embodiments, a gap filter is applied to fill gaps in the peak list. In some embodiments, the parameters for building a chromatogram comprise a minimum group size of 15 scans, group intensity threshold of 1000, a minimum highest intensity of 1000, and an m/z tolerance of 0.0 m/z or 20.0 ppm.

In some embodiments, the number of peaks detected by data processing is at least 5, 10, 15, 20, 25, 30, 35, 40, 45, 50, 75, 100, 125, 150, 175, 200, 225, 250, 300, 350, 400, 450, 500, or 1000 peaks per spent nutrient media sample. In some embodiments, the number of peaks identified by data processing is at or around 100 peaks per spent nutrient media sample.

In some embodiments, the chromatograms are then visualized. The peaks are plotted on a two-dimensional plot where the x-axis is the m/z ratio and the y-axis is the peak intensity. Methods of visualizing MS data using the software exemplified in the present disclosure (e.g., MZmine 2) are described in Katajamaa and Oresic, BMC Bioinformatics, 2005 Jul. 18, 6:179; Lee et al., European Symposium on Artificial Neural Networks, 2000, pg. 13-20; and Sammon et al., IEEE Trans, Comp,, 1969;C-18:401-409.

### iv) Chromatogram Alignment

Now that consensus peaks have been identified for each spent nutrient media sample, the next step is to align the peaks between samples. Peaks from the same compound usually match closely in m/z values, but there can be variation in retention times between the runs, These factors depend on mass accuracy and resolution of the mass spectrometer and the analytical method used. Software for processing MS data (e.g., MZmine 2) implement various statistical methods for alignment of mass spectra data sets (Katajamaa and Oresic. BMC Bioinformatics, 2005 Jul. 18; 6:179), In some embodiments, the parameters for aligning peaks between samples comprise an m/z tolerance of 0.0 m/z or 20.0 ppm, m/z weight of 1, a retention time tolerance of 0.15 min, and a retention time weight of 1.

The resulting processed MS data are saved and referred to as a “chemical spectra” of spent nutrient media from small lab-scale culture, or a “metabolite fingerprint” associated with a particular microorganism. In some embodiments, the processed chemical spectra data (from e.g., one or more ladder strains) is then used to generate a predictive model of phenotypic performance of a host cell in industrial culture (e.g., a culture with a nutrient media volume of 0.25 L or greater). In other embodiments, the processed chemical spectra data (from e.g., a test strain or independent host cell) is provided to a model in order to predict phenotypic performance of a host cell in industrial culture.

A non-limiting list of exemplary data analysis tools that can process and statistically analyze mass spectrometry data, and perform the steps described above are provided in Table 1 below.

TABLE 1
Mass Spectrometry Software

| Name | Description | References |
|---|---|---|
| MassHunter | Mass spectrometry screening software by | agilent.com/en/products/ |
| Qualitative Data | Agilent Technologies for the confirmation of | software-informatics/mass- |
| Analysis | target or suspect compounds, and for the | spectrometry- |
| | identification of unknown analytes. | software/data- |
| | | analysis/qualitative- |
| | | analysis; Accessed: Apr. |
| | | 29, 2020. |
| MassHunter | Software by Agilent Technologies optimized for | |
| Profinder | batch feature extraction from TOF and Q-TOF | |
| | based profiling data files and MSD data files | |
| Proteowizard | Open-source and cross-platform tools and | Kessner et al., |
| | libraries for proteomic data analyses. It provides | Bioinformatics, 24(21): |
| | a framework for unified mass spectrometry data | 2534-6; Chambers et al., |
| | file access and performs standard chemistry and | Nat Biotechnol. |
| | LCMS dataset computations. | 30(10): 918-20. |
| MSconvert | Command line tool in ProteoWizard software for | Kessner et al., |
| | converting between various file formats | Bioinformatics, 24(21): |
| | | 2534-6; Chambers et al., |
| | | Nat Biotechnol. |
| | | 30(10): 918-20. |
| JMP | Software for statistical analyses of data | jmp.com; Accessed: Apr. |
| | | 29, 2020. |
| MarkerView | Commercial software for statistical analysis for | Ciborowski et al., |
| Software | quantitative mass spectrometry data sets from | Proteomic Profiling and |
| | metabolomics and proteomic profiling | Analytical Chemistry: The |
| | applications. | Crossroads, 2016. |
| Mascot Distiller | Software by Matrix Science for peak picking | Bollineni et al. Scientific |
| | and raw data preprocessing. Optional toolbox | Reports. 2018; 8: 2117; |
| | for label-free quantification as well as isobaric | Koenig et al., J Proteome |
| | labeling and isotopic labeling. Supports raw file | Res. 2008; 7(9): 3708-17.; |
| | formats from all major instrument vendors. | Perkins et al., |
| | | Electrophoresis. 1999; |
| | | 20(18): 3551-67. |
| Mascot Server | Supports quantification based on isobaric | Koenig et al., J Proteome |
| | labeling as long as all the required information is | Res. 2008; 7(9): 3708-17.; |
| | part of the MS/MS spectrum. | Perkins et al., |
| | | Electrophoresis. 1999; |
| | | 20(18): 3551-67. |
| MassChroQ | Peptide quantification analysis of label free or | Valot et al. Proteomics. |
| | various isotopic labeling methods (e.g., SILAC, | 2011; 11(17): 3572-7. |
| | ICAT, N-15, and C-13), works with high and | |
| | low resolution spectrometer systems, supports | |
| | complex data treatments as peptide or protein | |
| | fractionation prior to MS analysis (e g., SCX, | |
| | SDS-PAGE). | |
| MaxQuant | Quantitative proteomics software that allows the | Tyanova et al. Nature |
| | analysis of label free and SILAC based | Protocols. 2016; 11: 2301- |
| | proteomics experiments. | 2319. |
| MultiQuant | Processes quantitative data sets from TripleTOF | Available at: |
| Software | or QTRAP systems, including MRM | sciex.com/products/software/ |
| | and SWATH Acquisition. | multiquant-software. |
| | | Accessed: 24 Mar. 2020. |
| OpenMS/ | Software C++ library for LC-MS/MS data | Rost et al., Nat. Methods. |
| TOPP | management and analysis that offers an | 2016; 13 (9): 741-8. |
| | infrastructure for the development of mass | |
| | spectrometry related software. Allows peptide | |
| | and metabolite quantification, supporting label- | |
| | free and isotopic-label based quantification | |
| | (such as iTRAQ and TMT and SILAC) as well | |
| | as targeted SWATH-MS quantification. | |
| ProtMax | ProtMAX is a software tool for analyzing | Egelhofer et al., Nat |
| | shotgun proteomics mass spectrometry data sets. | Protoc.. 2013; 8(3): 595- |
| | | 601. |
| Spectronaut | Commercial software for quantitative | Law, K P. Expert Rev |
| | proteomics based on the mProphet algorithm that | Proteomics. 2013; |
| | allows the targeted analysis of data independent | 10(6): 551-566. |
| | acquisition (DI) data sets for label-free peptide | |
| | quantitation, also called SWATH acquisition. | |
| Skyline | Open source software that supports building | Maclean, B. |
| | Selected Reaction Monitoring (SRM)/Multiple | Bioinformatics. 2010; |
| | Reaction Monitoring (MRM), Parallel Reaction | 26(7): 966-968. |
| | Monitoring (PRM - Targeted MS/MS), Data | |
| | Independent Acquisition (DIA/SWATH) and | |
| | targeted DDA with MS1 quantitative methods | |
| | and analyzing the resulting mass spectrometer | |
| | data. | |
| SWATH | Commercial software processing tool within | Navarro et al. Nature |
| Software 2.0 | Peak View that allows targeted data processing | Biotechnology. 2016; |
| | of SWATH acquisition data. Using a | 34(11): 1130-1136. |
| | protein/peptide ion library, fragment ion | |
| | extracted ion chromatograms (XICs) are | |
| | generated, scored and quantified for peptides | |
| | from the library. After false discovery rate | |
| | analysis, results are filtered and quantitative | |
| | peptide/protein data can be exported for | |
| | statistical analysis. | |
| BACIQ | Software that integrates peptide intensities and | Peshkin et al. Molecular |
| | peptide-measurement agreement into confidence | & Cellular Proteomics. |
| | intervals for protein ratios. | 2019, |
| | | mcp.TIR119.001317. |
| TopFD | Software tool for top-down spectral | proteomics.informatics. |
| | deconvolution and a successor to MS-Deconv. It | iupui.edu/software/topfd/. |
| | groups top-down spectral peaks into isotopomer | Accessed: 24 Mar. 2020. |
| | envelopes and converts isotopomer envelopes to | |
| | monoisotopic neutral masses. It also extracts | |
| | proteoform features from LC-MS or CE-MS | |
| | data. | |
| ArtIST by | MALDI-TOF MS data analysis and biomarker | cloverbiosoft.com/ |
| Clover Biosoft | discovery tools, based on artificial intelligence | Accessed: 24 Mar. 2020. |
| | and machine learning algorithms. ArtIST is an | |
| | online service. | |
| Advanced | Commercial solutions for the interpretation of | acdlabs.com/products/adh/ms/ |
| Chemistry | MS and xC/MS data with spectrum/structure | Accessed: 24 Mar. 2020. |
| Development | matching, identification of known and unknown | |
| | metabolites, as well as for the identification of | |
| | compounds through spectral comparison. | |
| AnalyzerPro | Software application from SpectralWorks for | spectralworks.com/products/ |
| | processing mass spectrometry data. It can | analyzerpro/ |
| | process both GC-MS and LC-MS data using | Accessed: 24 Mar. 2020. |
| | qualitative and quantitative data processing and | |
| | is used in metabolomics using MatrixAnalyzer | |
| | for the comparison of multiple data sets. | |
| | Recently extended to include statistical analysis | |
| | and visualisation tools (PCA). | |
| DeNovoGUI | Software with a graphical user interface for | Muth et al., Journal of |
| | running parallelized versions of the freely | Proteome Research. 2014; |
| | available de novo sequencing software tools | 13(2): 1143-1146. |
| | Novor and PepNovo+. | |
| [El-MAVEN] | Software for processing labeled LC-MS, GC-MS | Sahil et al., |
| | and LC-MSZMS data in open-formats (mzXML, | ElucidataInc/ElMaven: |
| | mzML, CDF). The software has a graphical and | v0.6.1. doi: |
| | command line interface with integration to a | 10.5281/zenodo.2537593 |
| | cloud platform for storage and further analy ses | |
| | like relative flux and quantification. | |
| ESIprot | Enables the charge state determination and | Winkler, R. Rapid |
| | molecular weight calculation for low resolution | Communications in Mass |
| | electrospray ionization mass spectrometry data | Spectrometry. 2010; |
| | of proteins. | 24(3): 285-94. |
| KnowItAll | Software from Bio-Rad Laboratories, Inc. with | bio-rad.com/en- |
| Spectroscopy | solutions for mass spectrometry including: | us/product/knowitall-u- |
| Software & | spectral analysis, database searching (spectrum, | spectra-database-for-your- |
| Mass Spectral | structure, peak, property, etc.), processing, | campus?ID=NH26LO8UU |
| Library | database building (MS or multiple techniques | Accessed: 24 Mar. 2020 |
| | including IR, Raman, NMR, UV, | |
| | Chromatograms), spectral subtraction, plus tools | |
| | for reporting and ChemWindow structure | |
| | drawing. | |
| LabSolutions | Software by Shimadzu Corporation used with | ssi.shimadzu.com/products/ |
| LCMS | mass spectrometry and HPLC instruments. | liquid-chromatography- |
| | | mass-spectrometry/lcms- |
| | | software.html |
| | | Accessed: 24 Mar. 2020. |
| Mass++ | Analysis software for mass spectrometry that | Tanaka et al. J. Proteome |
| | can import and export files with open-formats | Res. 2014; 13(8): 3846- |
| | (mzXML, mzML) and load some instrument | 3853. |
| | vendor formats; users can develop and add | |
| | original functions as Mass++ plug-ins. | |
| MassMap | General-purpose software suite for automated | massmap.de/ |
| | evaluation of MS data by MassMap GmbH & | Accessed: 24 Mar. 2020. |
| | Co. KG, suitable for LC/MS and GC/MS data of | |
| | all kinds of molecules, the analysis of intact | |
| | mass spectra of proteins, the analysis of general | |
| | HDX experiments and the HDX fragment | |
| | analysis of peptides, with particular method for | |
| | the identification of unexpected/unknown | |
| | components in even very complex mixtures. | |
| Mass-Up | Supports the preprocessing and analysis of | Lopez-Fernandez et al., |
| | MALDI-TOF mass spectrometry data that loads | BMC Bioinformatics. |
| | data from mzML, mzXML and CSV files and | 2015; 16: 318. |
| | allows users to apply baseline correction, | |
| | normalization, smoothing, peak detection and | |
| | peak matching. In addition, it allows the | |
| | application of different machine learning and | |
| | statistical methods to the preprocessed data for | |
| | biomarker discovery, unsupervised clustering | |
| | and supervised sample classification. | |
| mineXpert | Graphical user interface-based software for mass | Rusconi, F. J. Proteome |
| | spectral data visualization/mining. Supports ion | Res. 2019; 18(5): 2254- |
| | mobility mass spectrometry. A program of | 2259. |
| | the msxpertsuite.org software suite. | |
| massXpert | Graphical user interface-based (GUI) software | Rusconi, F. |
| | for simulating and analyzing mass spectrometric | Bioinformatics. 2009; |
| | data obtained on known bio-polymer sequences. | 25(20): 2741-2.; |
| mMass | Multi-platform package of tools for mass | mmass.org/ |
| | spectrometric data analysis and interpretation | Accessed: 24 Mar. 2020. |
| | written in Python. | |
| MSight | Software formass spectrometry imaging | Palagi et al. Proteomics. |
| | developed by the Swiss Institute of | 2005; 5(9): 2381-4. |
| | Bioinformatics. | |
| MSiReader | Vendor-neutral interface built on the Matlab | Robichaud et al. J of |
| | platform designed to view and perform data | American Society for |
| | analysis of mass spectrometry imaging | Mass Spectrometry. 2013; |
| | data. Matlab is not required to use MSiReader. | 24(5): 718-721. |
| mspire | Mass spectrometry toolbox written in ruby that | Prince, J T. |
| | includes an mzML reader/writer, in-silico | Bioinformatics. 2008; |
| | digestion and isotopic pattern calculation etc.; | 24(23): 2796-2797 |
| | submodules such as mspire-lipidomics, mspire- | |
| | sequest, and mspire-simulator extend the | |
| | functionality. | |
| Multimaging | Software for mass spectrometry imaging | imabiotech.com/ |
| | designed to normalize, validate and interpret MS | multimaging-cloud/ |
| | images. | Accessed: 24 Mar. 2020. |
| multiMS- | ms-alone and multiMS-toolbox is a tool chain | Cejar et al. Rapid |
| toolbox | for mass spectrometry data peak extraction and | Commun Mass Spectrom. |
| | statistical analysis. | 2018; 32(11): 871-881. |
| mzCloud | Web-based mass spectral database that | mzcloud.org/ |
| | comprises a collection of high and low | Accessed: 24 Mar. 2020. |
| | resolution tandem mass spectrometry data | |
| | acquired under a number of experimental | |
| | conditions. | |
| MZmine | An open-source software for mass-spectrometry | Katajamaa and Oresic, |
| | data processing and analysis. | BMC Bioinformatics. |
| | | 2005 Jul. 18; 6: 179. |
| MZmine 2 | An open-source software for mass-spectrometry | Katajamaa et al., |
| | data processing and analysis. | Bioinformatics. 2006 Mar. |
| | | 1; 22(5): 634-6. |
| OmicsHub | OmicsHub Proteomics combines a LIMS for | .Lloret et al. J Biomol |
| Proteomics | mass spec information management with data | Tech. 2010; 21(3 Suppl): |
| | analysis functionalities on one platform. | S21. |
| OpenChrom | Chromatography and mass spectrometry | Wenig et. al. BMC |
| | software that can be extended using plug-ins and | Bioinformatics. 2010, |
| | is available for several operating systems | 11: 405. |
| | (Microsoft Window's, Linux, Unix, Mac OS X) | |
| | and processor architectures (x86, x86_64, ppc). | |
| | with converters for the native access of various | |
| | data files, e.g. converters for mzXML, netCDF, | |
| | Agilent, Finnigan and Vartan file formats. | |
| ORIGAMI | Software suite for analysis of mass spectrometry | Migas et al. International |
| | and ion mobility mass spectrometry datasets. | Journal of Mass |
| | ORIGAMI was originally developed to improve | Spectrometry. 2018; |
| | the analysis workflows of activated IM- | 427: 20-28. |
| | MS/collision induced unfolding (CIU) datasets | |
| | and allow seamless visualisation of results. | |
| | Recently, ORIGAMI was modified to be more | |
| | accepting of non-MS centric and enables | |
| | visualisation of results from other sources as | |
| | well as enables exporting of all results in an | |
| | interactive format where the user can share any | |
| | dataset and visualize in an internet browser. | |
| PatternLab | Software for post-analysis of SEQUEST, | Carvalho et al. BMC |
| | ProLuCID or Comet database search results | Bioinformatics. 2008; |
| | filtered by DTA Select or Census. | 9: 316. |
| pyOpenMS | Open-source Python library for mass | Rost et al. Proteomics. |
| | spectrometry, specifically for the analysis of | 2014; 14(1): 74-7. |
| | proteomics and metabolomics data in Python. | |
| PeakInvestigator | 3-4X effective resolution improvement in post- | veritomyx.com/ |
| | processing of raw profile data output from mass | PeakInvestigator.php |
| | specs. Veritomyx advanced signal processing | Accessed: 24 Mar. 2020. |
| | software for peak detection, deconvolution, and | |
| | centroiding of raw profile mass spec data reveals | |
| | multiple peaks hidden in overlapped data. | |
| | Notable features: order-of-magnitude | |
| | improvements in mass and abundance precision | |
| | for deconvolved peaks; local dynamic | |
| | baselining; advanced thresholding algorithm | |
| | increases sensitivity across wide dynamic range; | |
| | statistically-driven and completely automated | |
| | (no user-to-user variation). More complete and | |
| | precise resulting mass lists facilitate faster and | |
| | cost-efficient subsequent determination of | |
| | correct biomolecular identifications. | |
| Pinnacle | Quantitation of proteins across hundreds of | Prakash et al., J Proteome |
| | samples using DDA, DIA, PRM or SRM with | Res. 2014; 13: 5415-5430.; |
| | fully integrated statistics and biological | Stewart et al., Proteomics. |
| | interpretation, to complete N-linked | 2017; 17(6): |
| | glycoprotein identification routine, to a very in- | 10.1002/pmic.201600300 |
| | depth analysis in protein characterization, | |
| | including peptide mapping, error tolerant search | |
| | and disulfide analysis, all of this is available in a | |
| | single software. | |
| PIQMIe | Web-based tool that aids in reliable and scalable | Kuzniar and Kanaar. |
| | data management, analysis and visualization of | Nucleic Acids. 2014; |
| | semi-quantitative (SILAC) proteomics | 42(W1): W100-W106. |
| | experiments. | |
| ProMass | Automated biomolecule deconvolution and | enovatia.com/products/ |
| | reporting software package that is used to | promass/ |
| | process ESI/LC/MS data or single ESI mass | Accessed: 24 Mar. 2020. |
| | spectra. It uses the novel deconvolution | |
| | algorithm, ZNova, to produce artifact-free | |
| | deconvoluted mass spectra. ProMass is currently | |
| | available for Thermo, Waters, and Shimadzu | |
| | platforms. It is also available in a “lite” browser- | |
| | based format called ProMass for the Web that | |
| | does not require any installation or software | |
| | download. | |
| Proteomatic | Data processing pipeline created for the purpose | Specht et al. |
| | of evaluating mass spectrometric proteomics | Bioinformatics. 2011; |
| | experiments. | 27(8): 1183-1184. |
| ProteomicsTools | Software for the post-analysis of MASCOT, | Sheng et al., J Proteome |
| | SEQUEST, Comet, XTandem, PFind, | Res. 2012; 11(3): 1494- |
| | PeptidePhophet, MyriMatch, MSGF, OMSSA, | 502. |
| | MSAmanda or Percolator database search result. | |
| ProteoWizard | Link library and tools that are a set of modular | Chambers et al Nat |
| | and extensible open-source, cross-platform tools | Biotechnology. 2012; |
| | and software libraries that facilitate proteomics | 30: 918. |
| | data analysis. | |
| ProteoWorker | Cloud-based software for proteomics data | proteoworker.com/ |
| | analysis including COMET, Peptide Prophet, | Accessed: 24 Mar. 2020. |
| | ProteinProphet and extensive data sorting, | |
| | filtering and annotation tools. | |
| pymzML | Python module to interface mzML data in | Bald et al. Bioinformatics. |
| | Python based on cElementTree with additional | 2012; 28(7): 1052-3. |
| | tools for MS-informatics. | |
| Pyteomics | A Python framework for proteomics data | Goloborodko et al. J Am |
| | analysis. | Soc Mass Spectrum. 2013; |
| | | 24(2): 301-4. |
| Quantinetix | Software for mass spectrometry imaging | imabiotech.com/Quantinetix- |
| | designed to quantify and normalize MS images | TM-Maldi- |
| | in various study types that is compatible with a | Imaging.html?lang=en |
| | variety of MSI instalments, including Bruker, | Accessed 24 Mar. 2020. |
| | Sciex, Thermo and with iMZML. | |
| Scaffold | Suite of proteomics tools for analyzing spectra, | Searle B C. Proteomics. |
| | peptides and proteins across multiple samples. | 2010: 10(6): 1265-9. |
| SCIEX OS | Next generation software by SCIEX controlling | sciex.com/products/software/ |
| | the X-series mass spectrometers and support for | sciex-os-software |
| | data analysis acquired using the Analyst | Accessed: 24 Mar. 2020. |
| | software suite. | |
| SCiLS Lab | Statistical analysis of MALDI imaging mass | scils.de/ |
| | spectrometry data that integrates with Bruker | Accessed: 24 Mar. 2020. |
| | MALDI imaging. | |
| SIMION | Ion optics simulation program | Silva et al. Vacuum. 2019; |
| | | 164: 300-307. |
| Spectrolyzer | Software that provides bioinformatics data | Zucht et al. Combinatorial |
| | analysis tools for different mass spectrometers | Chemistry & High |
| | that focuses on finding protein biomarkers and | Throughput Screening. |
| | detecting protein deviations. | 2005; 8(8): 717-23. |
| Spectromania | Software for analysis and visualization of mass | pxbiovision.com/downloads/ |
| | spectrometric data. | downloads/index.html |
| | | Accessed: 24 Mar. 2020. |
| Trans-Proteomic | Collection of integrated tools for MS/MS | Deutsch et al. Proteomics. |
| Pipeline (TPP) | proteomics that includes PeptideProphet for the | 2010; 10(6): 1150-1159. |
| | statistical validation of peptide-spectra-matches | |
| | using search engine results. | |
| VIPER | Analysis of accurate mass and chromatography | Monroe, M E. |
| | retention time analysis of LC-MS features | Bioinformatics. 2007; |
| | (accurate mass and time tag approach). | 23: 2021-3. |
| Xcalibur ™ | Software by Thermo Fisher Scientific used with | thermofisher.com/order/ |
| | mass spectrometry instruments. | catalog/product/OPTON- |
| | | 30965#/OPTON-30965. |
| XCMS | Metabolomic and lipidomic data processing | Gowda et al. Analytical |
| Online (Cloud- | platform | Chemistry. 2014; |
| Based) | | 86(14): 6931-9. |

In some embodiments, the MS analytical software is MZmine 2. MZmine 2 is an open-source data processing software toolbox for the processing and visualization of mass spectrometry based on molecular profile data. This software contains methods for all data processing stages preceding differential analysis: spectral filtering, mass detection, and peak alignment. MZmine 2 also implements a recursive peak search algorithm and a secondary peak picking method for improving already aligned results, as well as a normalization tool that uses multiple internal standards. Visualization tools enable comparative viewing of data across multiple samples. The software is freely available under the GNU General Public License and it can be obtained at: mzmine.sourceforge.net. The MZmine 2 software is described in Katajarnaa and Oresic, BMC Bioinformatics, 2005 Jul. 18; 6:179, herein incorporated by reference in its entirety.

In some embodiments, the MS analytical software is MZmine 2. The MZmine 2 software includes additional features such as support for the mzXML data format, capability to perform batch processing for a large number of files, support for parallel processing, methods for calculating peak areas using post-alignment peak picking algorithm and implementation of Sammon's mapping and curvilinear distance analysis for data visualization and exploratory analysis. The software is freely available under the GNU General Public License and it can be obtained at http//mzmine.sourceforge.net. The MZmine software is described in Katajamaa et al, Bioinformatics, 2006 Mar. 1; 22(5):634-6, herein incorporated by reference in its entirety.

In some embodiments, the present disclosure also teaches additional steps that may be conducted as part of the methods of the present disclosure. In some embodiments, these methods are optional. An optional stage in data processing is smoothing, which can be left out if the data is not noisy or if the input data is already available as centroids. Smoothing aims to remove noise in the measured spectra, which facilitates further peak detection. Spectra can be smoothed by implementing moving average, Gaussian, or Savitzky-Golay filters. Choice of methods for smoothing depends on the nature of input data and can easily be determined by one of skill in the art.

Another additional step in data processing is normalization. The term “normalization” refers to methods used to correct for differences in the total amount of protein desorbed and ionized from the sample plate. In some embodiments, the mass spectral data is normalized to reduce systematic error. The analytical software exemplified in the present disclosure (e.g., MZmine 2) is capable of performing normalization of the mass spectra data.

After data processing (e.g., mass detection, chromatogram building, and peak alignment), the data can be further analyzed with packages such as MP, MatLab (MathWorks, Inc.) or R Statistical Language, which have a large collection of tools available for statistical analysis of multivatiate data.

### Detection and Quantitation of Metabolites by Other Analytical Instruments:

In some embodiments, the present disclosure teaches that other analytical instruments may be used to detect one or more metabolites in the spent nutrient sample in order to predict phenotypic performance of a host cell. These instruments include, but are not limited to, Fourier transform infrared (FTIR) spectroscopy, Raman spectroscopy, and nuclear magnetic resonance (NMR) spectroscopy.

FTIR spectroscopy is a technique that uses infrared light to observe properties of a solid, liquid, or gas. It measures the absorption, emission, and photo-conductivity of matter by shining a narrow beam of infrared light at the matter in various wavelengths and detecting how the matter responds to each wavelength. Once the data has been Obtained, it is converted into digital information using a mathematical algorithm known as the “Fourier transform” to identify molecular components and structures.

Raman spectroscopy is a technique used to determine vibrational modes of molecules, which provides a structural fingerprint by which the molecules can be identified. Raman spectroscopy relies upon inelastic scattering of photons, known as Raman scattering. A source of monochromatic light, usually from a laser in the visible, near infrared, or near ultraviolet range is used, although X-rays can also be used. The laser light interacts with molecular vibrations, phonons or other excitations in the system, resulting in the energy of the laser photons being shifted up or down. The shift in energy gives information about the vibrational modes in the system. See, Gardiner, D. J. (1989). Practical Raman Spectroscopy.

NMR spectroscopy is a technique used to observe local magnetic fields around atomic nuclei. The sample is placed in a magnetic field and the NMR signal is produced by excitation of the nuclei sample with radio waves into nuclear magnetic resonance, which is detected with sensitive radio receivers. The intramolecular magnetic field around an atom in a molecule changes the resonance frequency, thus giving access to details of the electronic structure of a molecule and its individual functional groups. Besides identification, NMR spectroscopy provides detailed information about the structure, dynamics, reaction state, and chemical environment of molecules. The most common types of NMR are proton and carbon-13 NMR spectroscopy, but it is applicable to any kind of sample that contains nuclei possessing spin.

### Generating a Predictive Model of Phenotypic Performance

In some embodiments, the methods of the present disclosure generate a predictive model of phenotypic performance that can be applied to the metabolite fingerprint of an independent host cell to predict the performance of the independent host cell in industrial culture.

The term “predicting”, “predict”, or “predictive” as used herein, or in the narrower sense, the phrase “predicting phenotypic performance of host cells in industrial cultures,” means that the phenotype of the host cell in large-scale development is anticipated. This anticipation is based on the metabolite fingerprint of a particular host cell in small lab-scale culture at the point in time when the methods of the present disclosure are applied. As such, said point in time is temporally earlier than the point in time corresponding to the future phenotype of interest which is being predicted.

In some embodiments, the predictive model of the present disclosure is based on a training dataset. in some embodiments, the training dataset is a “strain ladder”. The term “strain ladder” refers to a collection of strains, each with different measurements of performance (e.g., in industrial culture) and an associated metabolite fingerprint for each of those strains (e.g. from a small lab-scale culture). In some embodiments, the metabolite fingerprinting variable and the phenotypic performance variable for a strain ladder are used to generate a transfer function for the prediction of phenotypic performance of independent or “unknown” strains.

In some embodiments, the predictive model of the present disclosure utilizes an anchor or base strain to generate the transfer function for the prediction of phenotypic performance of independent or “unknown” strains. The term “anchor” or “base” strain refers to a host strain exhibiting a basal level of performance, typically significantly lower than other engineered strains. In some embodiments, the anchor or base strain generates a transfer function that more accurately predicts phenotypic performance of host cells in industrial cultures than a transfer function generated without an anchor or base strain.

In some embodiments, the predictive model is based on a “metabolite fingerprinting variable” and a “phenotypic performance variable.” The metabolite fingerprint variable is based on chemical spectra of a plurality of spent nutrient media with each spent nutrient media sample having been derived from small lab-scale culture of a host cell exhibiting a known phenotypic performance measurement. In some embodiments, the metabolite fingerprint of a host cell in small lab-scale culture is associated with the phenotypic performance measurement of a host cell in industrial culture.

In some embodiments, the metabolite fingerprint variable comprises data from at least 5, 10, 15, 20, 25, 30, 35, 40, 45, 50, 75, 100, 125. 150, 175, 200, 225. 250, 300, 350, 400, 450, 500, or 1000 chemical spectra.

In some embodiments, the predictive model is based on a phenotypic performance output variable. in some embodiments, the phenotypic performance variable comprises information related to the phenotypic performance of a microorganism in industrial culture. Thus, in some embodiments, the present disclosure teaches training datasets that comprise metabolite fingerprints from small lab-scale cultures of selected microorganisms, and phenotypic performance data from the same microorganism in industrial (e.g. bench-scale) cultures.

In some embodiments, phenotypic performance variable in the training dataset comprises yield data of one or more products of interest. In some embodiments, the phenotypic performance variable is based on the production or yield of one or more products of interest in industrial culture. The term “yield” is defined as the amount of product obtained per unit weight of raw material and may be expressed as gram of product per gram of substrate (gig). Yield may be expressed as a percentage of the theoretical yield. Products of interest include, but are not limited to, a small molecule, enzyme, protein, peptide, amino acid, organic acid, synthetic compound, fuel, alcohol, primary extracellular metabolite, secondary extracellular metabolite, intracellular component molecule, and combinations thereof. Persons having skill in the art will appreciate that, in some embodiments, the phenotypic performance variable can be another measure of phenotypic performance, including volumetric productivity, specific productivity, time to desired concentration, titre of a product of interest.

Table 2 below presents a non-limiting list of the product categories, biomolecules, and host cells, included within the scope of the present disclosure. These examples are provided for illustrative purposes, and are not meant to limit the applicability of the presently disclosed technology.

TABLE 2
Illustrative Host Cells and Products of Interest

| Product category | Products | Host category | Hosts |
|---|---|---|---|
| Amino acids | Lysine | Bacteria | Corynebacterium glutamicum |
| Amino acids | Methionine | Bacteria | Escherichia coli |
| Amino acids | MSG | Bacteria | Corynebacterium glutamicum |
| Amino acids | Threonine | Bacteria | Escherichia coli |
| Amino acids | Threonine | Bacteria | Corynebacterium glutamicum |
| Amino acids | Tryptophan | Bacteria | Escherichia coli |
| Enzymes | Enzymes (11) | Filamentous fungi | Trichoderma reesei |
| Enzymes | Enzymes (11) | Fungi | Myceliopthora thermophila (C1) |
| Enzymes | Enzymes (11) | Filamentous fungi | Aspergillus oryzae |
| Enzymes | Enzymes (11) | Filamentous fungi | Aspergillus niger |
| Enzymes | Enzymes (11) | Bacteria | Bacillus subtilis |
| Enzymes | Enzymes (11) | Bacteria | Bacillus licheniformis |
| Enzymes | Enzymes (11) | Bacteria | Bacillus clausii |
| Flavor & Fragrance | Agarwood | Yeast | Saccharomyces cerevisiae |
| Flavor & Fragrance | Ambrox | Yeast | Saccharomyces cerevisiae |
| Flavor & Fragrance | Nootkatone | Yeast | Saccharomyces cerevisiae |
| Flavor & Fragrance | Patchouli oil | Yeast | Saccharomyces cerevisiae |
| Flavor & Fragrance | Saffron | Yeast | Saccharomyces cerevisiae |
| Flavor & Fragrance | Sandalwood oil | Yeast | Saccharomyces cerevisiae |
| Flavor & Fragrance | Valencene | Yeast | Saccharomyces cerevisiae |
| Flavor & Fragrance | Vanillin | Yeast | Saccharomyces cerevisiae |
| Food | CoQ10/Ubiquinol | Yeast | Schizosaccharomyces pombe |
| Food | Omega 3 fatty acids | Microalgae | Schizochytrium |
| Food | Omega 6 fatty acids | Microalgae | Schizochytrium |
| Food | Vitamin B12 | Bacteria | Propionibacterium freudenreichii |
| Food | Vitamin B2 | Filamentous fungi | Ashbya gossypii |
| Food | Vitamin B2 | Bacteria | Bacillus subtilis |
| Food | Erythritol | Yeast-like fungi | Torula coralline |
| Food | Erythritol | Yeast-like fungi | Pseudozyma tsukubaensis |
| Food | Erythritol | Yeast-like fungi | Moniliella pollinis |
| Food | Steviol glycosides | Yeast | Saccharomyces cerevisiae |
| Hydrocolloids | Diutan gum | Bacteria | Sphingomonas sp |
| Hydrocolloids | Gellan gum | Bacteria | Sphingomonas elodea |
| Hydrocolloids | Xanthan gum | Bacteria | Xanthomonas campestris |
| Intermediates | 1,3-PDO | Bacteria | Escherichia coli |
| Intermediates | 1,4-BDO | Bacteria | Escherichia coli |
| Intermediates | Butadiene | Bacteria | Cupriavidus necator |
| Intermediates | n-butanol | Bacteria | Clostridium acetobutylicum |
| | | (obligate anaerobe) | |
| Organic acids | Citric acid | Filamentous fungi | Aspergillus niger |
| Organic acids | Citric acid | Yeast | Pichia guilliermondii |
| Organic acids | Gluconic acid | Filamentous fungi | Aspergillus niger |
| Organic acids | Itaconic acid | Filamentous fungi | Aspergillus terreus |
| Organic acids | Lactic acid | Bacteria | Lactobacillus |
| Organic acids | Lactic acid | Bacteria | Geobacillus thermoglucosidasius |
| Organic acids | LCDAs - DDDA | Yeast | Candida |
| Polyketides/Ag | Spinosad | Yeast | Saccharopolyspora spinosa |
| Polyketides/Ag | Spinetoram | Yeast | Saccharopolyspora spinosa |

In some embodiments, the phenotypic performance variable is the production of one or more proteins. In some embodiments, the phenotypic performance variable is the production of one or more metabolites. In some embodiments, the phenotypic performance variable is the production of one or more amino acids. In some embodiments, the phenotypic performance variable is the production of one or more vitamins. In some embodiments, the phenotypic performance variable is the production of one or more commodity chemicals. Numerous chemicals are known to be produced or known to be possible to produce in biological culture, such as ethanol, acetone, citric acid, propanoic acid, fumaric acid, butanol and 2,3-butanediol. See, e.g., Saxena, “Microbes in Production of Commodity Chemicals,” Applied Microbiology 2015: 71-81, incorporated by reference herein in its entirety. In some embodiments, the phenotypic performance measurement is production of one or more fine chemicals. In some embodiments, the phenotypic performance variable is the production of one or more specialty chemicals. In some embodiments, the phenotypic performance variable is the production of one or more pharmaceuticals. In some embodiments, the phenotypic performance variable is the production of one or more biofuels. In some embodiments, the phenotypic performance variable is the production of one or more biopolymers.

In some embodiments, the phenotypic performance variable is the production of one or more alcohols. Alcohols include, but are not limited to, ethanol, propanol, isopropanol, butanol, fatty alcohols, fatty acid esters, wax esters; hydrocarbons and alkanes such as propane, octane, diesel, JP8; polymers such as terephthalate, 1,3-propanediol, 1,4-butanediol, polyols, PHA, PHB, acrylate, adipic acid, ϵ-caprolactone, isoprene, caprolactam, rubber; commodity chemicals such as lactate, DHA, ϵ-hydroxypropionate, γ-valerolactone, lysine, serine, aspartate, aspartic acid, sorbitol, ascorbate, ascorbic acid, isopentenol, lanosterol, omega-3 DHA, lycopene, itaconate, 1,3-butadiene, ethylene, propylene, succinate, citrate, citric acid, glutamate, malate, HPA, lactic acid, THF, gamma butyrolactone, pyrrolidones, hydroxybutyrate, glutamic acid, levulinic acid, acrylic acid, malonic acid; specialty chemicals such as carotenoids, isoprenoids, itaconic acid; pharmaceuticals and pharmaceutical intermediates such as 7-ADCA/cephalosporin, erythromycin, polyketides, statins, paclitaxel, docetaxel, terpenes, peptides, steroids, omega fatty acids and other such suitable molecules of interest.

In some embodiments, the range of phenotypic performance measurements exhibited by a strain ladder comprises at least a 3, 4, 5, 6, 7, 8, 9, or 10-fold difference between the lowest and highest known phenotypic performance measurements.

### Multivariate Analysis Methods of Metabolite Fingerprint Variable and Phenotypic Performance Variable:

In some embodiments, the predictive model of phenotypic performance is constructed based on multivariate statistical analysis of the training dataset. Illustrative examples of multivariate statistical analyses that may be used to develop the predictive models of the present disclosure include, but are not limited to, partial least squares analysis (PLS), partial least squares discriminant analysis (PLS-DA), orthogonal partial least squares analysis (OPLS), or principal component analysis.

In some embodiments, partial least squares analysis is applied to the metabolite fingerprint and associated phenotypic performance measurement of a host cell. As used herein, “partial least squares analysis” or “PLS” refers to a statistical analysis known to those of ordinary skill in the art which can be used for quantitative predictions of phenotypic outcomes by finding a linear regression model.

In some embodiments, the predictive model of phenotypic performance is determined by partial least squares discriminate analysis (PLS-DA). The term “partial least squares discriminant analysis” or “PLS-DA” refers to the use of statistical analyses that discriminates between two or more naturally^{,} occurring groups. PLS-DA is known to those of skill in the art and may be utilized in certain embodiments of the present disclosure where qualitative predictions might be expected.

In some embodiments, orthogonal partial least squares analysis (OPLS) is applied to the metabolite fingerprint and associated phenotypic performance measurement of a host cell in order to generate a predictive model of phenotypic performance. As used herein, the term “orthogonal partial least squares analysis” or “OPLS” is a technique used to remove variation from X (descriptor variables) that is irrelevant to Y (quality variables, for example, yield). This type of analysis is described in Earlier, S. et al., Orthogonal signal correction of near-infrared spectra, Chemometrics and Intelligent Laboratory Systems, 44 (1998) 175-185 and U.S. Patent Publication No. US2003/0200040A1, herein incorporated by reference in their entirety.

In sonic embodiments, principal component analysis is applied to the metabolite fingerprint and associated phenotypic performance measurement of a host cell in order to generate a predictive model of phenotypic performance. As used herein, the term “principal component analysis” refers to a statistical analysis that transforms a number of possibly correlated variables into a smaller number of uncorrelated variables called principal components, wherein the first principal component accounts for as much of the variability in the data as possible, and each succeeding component accounts for as much of the remaining variability as possible. The process of using principal component analysis is within the ability of one of ordinary skill in the art.

The method of predicting phenotypic performance of a host cell may vary depending upon the embodiment of the disclosure used to establish the predictive model. For example, in some embodiments, the predictive model may allow for a quantitative prediction, wherein partial least squares analysis may be used to establish a linear correlation between the metabolite fingerprint and phenotypic performance of a host cell within a training set. Subsequently, the predictive model can then be applied to the metabolite fingerprint of an independent host cell in order to quantitatively predict phenotypic performance.

In some embodiments, the predictive model may allow for a qualitative prediction, wherein partial least squares discriminate analysis may be used to establish a correlation between the metabolite fingerprint and phenotypic performance within a training set. In partial least squares discriminate analysis, each host cell is assigned to a class, i.e., host cells that exhibit a particular phenotype and host cells that do not. Subsequently, the partial least squares discriminate analysis model can then be applied to the metabolite fingerprint of an independent host cell in order to predict which class the host cell will most closely resemble.

In some embodiments, outliers may be identified and excluded from the data set. As used herein, “outliers” means the infrequent observations or data points which do not appear to follow the characteristic distribution of the rest of the data. As such, outliers may greatly influence the slope of the regression line and the value of the correlation coefficient. Such outliers may be identified and excluded by statistical methods such as principal component analysis.

### Utilizing the Predictive Model of Phenotypic Performance

The methods of the present disclosure utilize a predictive model of phenotypic performance to determine the performance of an independent or “unknown” host cell in industrial culture (i.e., a host cell that was not included in the training dataset used to build the predictive model).

In some embodiments, the present disclosure teaches a method for predicting phenotypic performance of a host cell in industrial cultures, said method comprising the step of providing the chemical spectra of a first spent nutrient media from a small lab-scale culture to a predictive model. In some embodiments, the predictive model is based on multivariate analysis of the training dataset, as described above. In some embodiments, the chemical spectra of a first spent nutrient media is obtained by analyzing the spent nutrient media by mass spectrometry and mass spectrometry software. Methods of generating chemical spectra from a spent nutrient sample are described under the “sample collection” and “mass spectrometry” headings above.

As used herein, the term “independent host cell” refers to a host cell with unknown phenotypic performance in industrial cultures or a host cell that was not included in the training dataset used to develop the predictive model. Thus, in some embodiments, the present disclosure teaches that a spent nutrient sample will be obtained from the independent host cell and subjected to mass spectrometry analysis. The resulting chemical spectra from the spent nutrient sample will be used in a previously generated model to predict phenotypic performance of an independent host cell in industrial culture.

### Exemplary Workflow of Predicting Phenotypic Performance of Host Cells in Industrial Culture

This workflow illustrates the methods used to obtain a metabolite fingerprint from a series of host cells in small lab-scale culture (Le., cultures with a nutrient media volume ≤5 mL or 0.005 L). The metabolite fingerprint can then be utilized in a model to predict phenotypic performance in industrial culture (i.e., cultures with a nutrient media volume ≥0.25 L).

### Sample Collection

Grow the host cells of interest in small lab-scale culture under aseptic conditions. Separate the nutrient media from the host cells by centrifugation, thereby generating spent nutrient media, which is then used to predict phenotypic performance of the host cell.

### Sample Preparation and Mass Spectrometry

The chemical spectra of the spent nutrient sample is then determined using direct-injection electrospray ionization mass spectrometry (ESI-MS) and a time-of-flight (TOF) analyzer. To prepare the sample for ESI-MS, clarify, filter and dilute (e.g., 1:500 or 1:1000) the spent nutrient media sample to prevent contamination and to ensure that the chemical concentrations are within the dynamic range of the instrument. After preparation, inject one microliter of the sample into the mass spectrometer for metabolite analysis.

MS analyzes either positive or negative ions. The spent nutrient sample should be analyzed under both conditions in order to determine which ion mode generates the best predictive model. Acquire the MS data in centroid mode. Centroid mode compresses the data so that the mass-to-charge peaks have no width, only peak intensities. After all data is acquired for the spent nutrient sample, the data files are converted to the open-source file type .mzML using the msconvert application in ProteoWizard. The .mzmL files will then be processed and analyzed using MZmine 2.

### MS Data Processing and Analysis

MZmine 2 is MS analytical software used to perform mass detection, chromatogram building, and m/z peak alignment. Download and import the .mzML files from the mass spectrometer into MZmine 2.

Spectral Filtering: Select all files and view the total ion current (TIC) chromatograms. Identify and eliminate any samples that had poor injections.

Mass/Peak detection: View the number of scans obtained for each sample by clicking on the “+” symbol adjacent to each sample set. The typical range is 159-161 scans per sample. The number of scans per sample is important for mass detection.

Select all samples to be analyzed and then select mass detection. From the new panel, select “set filters”. Based on the number of scans identified above, enter the scan range (e.g., 1-161). Select a. retention time range where the primary peak resides on the TIC plot (e.g., 0.05-0.15 minutes). Set the MS level to 1, polarity to negative (but positive should also be analyzed as noted above), and the spectrum type as centroided. For the MS detector, select “centroid” and set the noise level to 1000. This is the peak intensity background subtraction. The sample can be previewed to show the peaks omitted based on this setting. Lastly, name the mass list to be generated by the mass detector. This will be the name of the peak lists generated for each scan/sample and what the chromatogram builder will use to generate a chromatogram for each sample.

Building the chromatogram for each sample: Select the ADAP chromatogram builder under the raw data methods and peak detection tabs. Set the filters to the same as the mass detection settings described above. Select the mass list created for mass detection. Set the minimum group size in number of scans (e.g., 15 scans). This means that the peaks of interest need to be in this number of scans (e.g., 15 scans) in order to qualify as part of the chromatogram. Next, set the m/z tolerance level (e.g., 0 m/z or 20 ppm). This setting determines which peaks in the scans are aggregated for the final chromatogram. If this setting is too wide, peaks may be incorporated that are not the discreet chemical. If the setting is too narrow, peaks may be missed that should have been grouped together. Lastly, add a suffix to identify each chromatogram build. After clicking “OK”, the screen will become populated with chromatograms for each sample. Expand a sample and a list of each identified m/z peaks should be present (e.g., 100 peaks per sample). The number of consensus peaks depends on a. number of factors (e.g., sample type, analytical method).

Peak Alignment: Now that consensus peaks have been identified for each sample, the peaks are aligned between samples. Select all samples of interest and then select “join aligner”. Create a peak list name. Set the m/z tolerance to the same setting as used in chromatogram building (e.g., 0 m/z or 20 ppm). The weight for m/z was set to 1 and the retention time tolerance was set to 0.15 minutes, which was the high retention time setting used for mass detection. After clicking “OK”, the aligned peak list should appear. Expand and examine the list of aligned m/z peaks across all samples.

Exporting the data to Excel: Highlight the aligned peak list. Select “Export to CSV file”. From the new window, create a filename. Check the following: Export row ID, Export row m/z, Export row number of detected peaks, and peak height (remember centroided peaks have no width, just height). Click “OK” to export.

Organizing the data in Excel: Open the .csv file. Organize the data in Excel so that each row represents a unique well_ID and each column represents a particular m/z peak area. Select all data and copy. Open a new table in the statistical software JMP. Paste with column names.

The JMP table can be joined by well ID to the data table that contains the strainID and, most importantly, the performance data associated with each strain. After joining datasets, each sample will have the associated strainID, m/z values, and performance data for Partial Least Squares analysis.

Developing a model to predict phenotypic performance: A transfer function will then be generated based in a metabolite fingerprint variable and an associated phenotypic performance variable using partial least squares analysis (PLS). PLS is useful when analyzing datasets with many X responses e.g., m/z values) and fewer Y responses (e.g., performance). Select the strains used to develop the transfer function, such as ladder strains or strains as part of a training dataset. Hide and exclude all other strains. Select PLS under the multivariate analysis tab. In the window, select all m/z values and deposit in the X factor field and then select all performance values and deposit in the Y factor field. Select “OK”.

Running PLS analysis: The PLS says that 15 factors gives the best fit with an R² value of 99.999, but this can be overfitting the function. The PLS analysis can be relaunched and constrained to a lower number of factors (e.g., 2 factors gives an R² value of 0.919049), The transfer function can then be plotted selecting the “Diagnostic Plots” tab.

Predicting phenotypic performance of independent strains: Now that the transfer function has been generated based on the training dataset, it can be used to predict phenotypic performance of independent “test” strains. Select “Save Prediction Formula”. On the data table, there will be a new column that has the predicted performance of all samples in the dataset. The predicted values can then be plotted for each strain for comparison.

Overall, this workflow demonstrates how metabolite fingerprinting of host cells can be used to predict phenotypic performance in industrial culture.

### Illustrative Host Cells of the Present Disclosure

Suitable host cells include, but are not limited to: bacterial cells, algal cells, plant cells, fungal cells, insect cells, and mammalian cells.

In some embodiments, suitable host cells include E. coli (e.g., SHuffle™ competent E. coli available from New England BioLabs in Ipswich, Mass.). In other embodiments, suitable host cells include microorganisms of the genus Corynebacterium. In some embodiments, Corynebacterium strains/species include: C. efficiens, with the deposited type strain being DSM44549; C. glutamicum, with the deposited type strain being ATCC13032; and C. ammoniagenes, with the deposited type strain being ATCC6871. Suitable host strains of the genus Corynebacterium, in particular of the species Corynebacterium glutamicum, are the known wild-type strains: Corynebacterium glutamicum ATCC13032, Corynebacterium acetoglutamicum ATCCC15806, Corynebacterium acetoacidophilum ATCC13870, Corynebacterium melassecola ATCC17965, Corynebacterium thermoaminogenes FERM BP-1539, Brevibacterium flavum ATCC14067, Brevibacterium lactofermentum ATCC13869, and Brevibacterium ATCC14020; and L-amino acid-producing mutants, or strains, prepared therefrom, such as, for example, the L-lysine-producing strains: Corynebacterium glutamicum FERM-P 1709, Brevibacterium flavum FERM-P 1708, Brevibacterium lactofermentum FERM-P 1712, Corynebacterium glutamicum FERM-P 6463, Corynebacterium glutamicum FERM-P 6464, Corynebacterium glutamicum DM58-1, Corynebacterium glutamicum DG52-5, Corynebacterium glutamicum DSM5714, and Corynebacterium glutamicum DSM12866.

In some embodiments, the host cell of the present disclosure is a eukaryotic cell. Suitable eukaryotic host cells include, but are not limited to, fungal cells, algal cells, insect cells, animal cells, and plant cells. Suitable fungal host cells include, but are not limited to, Ascomycota, Basidiomycota, Deuteromycota, Zygomycota, and Fungi imperfecti. Certain fungal host cells include yeast cells and filamentous fungal cells. Suitable filamentous fungi host cells include, for example, any filamentous forms of the subdivision Eumycotina and Oomycota, (see, e.g., Hawksworth et al., In Ainsworth and Bisby's Dictionary of The Fungi, 8^{th} edition, 1995, CAB International, University Press, Cambridge, UK, which is incorporated herein by reference). Filamentous fungi are characterized by a vegetative mycelium with a cell wall composed of chitin, cellulose and other complex polysaccharides. The filamentous fungi host cells are morphologically distinct from yeast.

In some embodiments, the filamentous fungal host cell may be a cell of a species of: Achlya, Acremonium, Aspergillus, Aureobasidium, Bjerkandera, Ceriporiopsis, Cephalosporium, Chrysosporium, Cochliobolus, Corynascus, Cryphonectria, Cryptococcus, Coprinus, Coriolus, Diplodia, Endothis, Fusarium, Gibberella, Gliocladium, Humicola, Hypocrea, Myceliophthora (e.g., Myceliophthora thermophila), Mucor, Neurospora, Penicillium, Podospora, Phlebia, Piromyces, Pyricularia, Rhizomucor, Rhizopus, Schizophyllum, Scytalidium, Sporotrichum, Talaromyces, Thermoascus, Thielavia, Tramates, Tolypocladium, Trichoderma, Volvariella, or teleomorphs, or anamorphs, and synonyms or taxonomic equivalents thereof In some embodiments, the filamentous fungus is selected from the group consisting of A. nidulans, A. ogzae, A. sojae, and Aspergilli of the A. niger.

Suitable yeast host cells include, but are not limited to: Candida, Hansenula, Saccharomyces, Schizosaccharomyces, Pichia, Kluyveromyces, and Yarrowia. In some embodiments, the yeast cell is Hansenula polymorpha, Saccharomyces cerevisiae, Saccaromyces carlsbergensis, Saccharomyces diasiaticus, Saccharomyces norbensis, Saccharomyces kluyveri, Schizosaccharomyces pombe, Pichia pastoris, Pichia finlandica, Pichia trehalophila, Pichia kodamae, Pichia membranaefaciens, Pichia opuntiae, Pichia thermotolerans, Pichia salictaria, Pichia quercuum, Pichia pijperr, Pichia stipitis, Pichia methanolica, Pichia angusta, Kluyveromyces lactis, Candida albicans, or Yarrowia lipolytica.

In certain embodiments, the host cell is an algal cell such as, Chlamydomonas (e.g., C. Reinhardtii) and Phormidium (P. sp. ATCC29409).

In some embodiments, the host cell is a prokaryotic cell. Suitable prokaryotic cells include gram positive, gram negative, and gram-variable bacterial cells. The host cell may be a species of, but not limited to: Agrobacterium, Alicyclobacillus, Anabaena, Anacystis, Acinetobacter, Acidothermus, Arthrobacter, Azobacter, Bacillus, Bifidobacterium, Brevibacterium, Butyrivihrio, Buchnera, Campestris, Camplyobacter, Clostridium, Corynebacterium, Chromatium, Coprococcus, Escherichia, Enterococcus, Enterobacter, Erwinia, Fusobacterium, Faecalibacterium, Francisella, Flavobacterium, Geobacillus, Haemophilus, Helicobacter, Klebsiella, Lactobacillus, Lactococcus, Hyobacter, Micrococcus, Microbacterium, Mesorhizobium, Methylobacterium, Methylobacterium, Mycobacterium, Neisseria, Pantoea, Pseudomonas, Prochlorococcus, Rhodobacter, Rhodopseudomortas, Rhodopseudomonas, Roseburia, Rhodospirillum, Rhodococcus, Scenedesmus, Streptomyces, Streptococcus, Synecoccus, Saccharomonospora, Saccharopolyspora, Staphylococcus, Serratia, Salmonella, Shigella, Thermoanaerobacterium, Tropheryma, Tularensis, Temecula, Thermosynechococcus, Thermococcus, Ureaplasma, Xanthomonas, Yersinia, and Zymomonas.

In some embodiments, the bacterial host cell is an industrial bacterial strain. Numerous industrial bacterial strains are known and suitable in the methods and compositions described herein.

In some embodiments, the bacterial host cell is of the Agrobacterium species (e.g., A. radiobacter, A. rhizogenes, A. rubi), the Arthrobacterspecies (e.g., A. aurescens, A. citreus, A. globformis, A. hydrocarboglutamicus, A. mysorens, A. nicotianae, A. pareineus, A. protophonniae, A. roseoparaffinus, A. sulfureus, A. ureafaciens), the Bacillus species (e.g., B. thuringiensis, B. anthracis, B. megaterium, B. subtilis, B. lentils, B. circulars, B. pludits, B. lautus, B. coagulans, B. brevis, B. firmus, B. alkaophius, B. lichemformis, B. clausii, B. stearothermophilus, B. halodurans and B. amyloliquefaciens. In some embodiments, the host cell will be an industrial Bacillus strain including but not limited to B. subtilis, B. pumilus, B. licheniformis, B. megaterium, B. clausii, B. stearothermophilus and B. amyloliquefaciens. In some embodiments, the host cell will be an industrial Clostridium species (e.g., C. acetobutylicum, C. tetani E88, C. litusehurense, C. saccharobutylicum, C. perfringens, C. beijerinckii). In some embodiments, the host cell will be an industrial Corynebacterium species (e.g., C. glutamicum, C. acetoacidophilum). In some embodiments, the host cell will be an industrial Escherichia species (e.g., E. coli). In some embodiments, the host cell will be an industrial Erwinia species (e.g., E. uredovora, E. carotovora, E. ananas, E. herbicola, E. punctata, E. terreus). In some embodiments, the host cell will be an industrial Pantoea species (e.g., P. citrea, P. agglomerans). In some embodiments, the host cell will be an industrial Pseudomonas species, (e.g., P. putida, P. aeruginosa, P. inevalonii). In some embodiments, the host cell will be an industrial Streptococcus species (e.g., S. equisimiles, S. pyogenes, S. uberis). In some embodiments, the host cell will be an industrial Streptomyces species (e.g., S. ambofaciens, S. achromogenes, S. avermitilis, S. coelicolor, S. aureofaciens, S. aureus, S. fungicidicus, S. griseus, S. lividans). In some embodiments, the host cell will be an industrial Zymininas species (e.g., Z. mobilis, Z. lipolytica), and the like.

The present disclosure is also suitable for use with a variety of animal cell types, including mammalian cells, for example. human (including 293, WI38, PER C6 and Bowes melanoma cells), mouse (including 3T3, NS0, NS1, Sp2/0), hamster (CHO, BHK), monkey (COS, FRhL, Vero), and hybridoma cell lines.

In various embodiments, strains that may be used in the practice of the disclosure, including both prokaryotic and eukaryotic strains, are readily accessible to the public from a number of culture collections such as American Type Culture Collection (ATCC), :Deutsche Sammlung von Mikroorganismen and Zellkulturen GmbH (DSM), Centraalbureau Voor Schimmelcultures (CBS), and Agricultural Research Service Patent Culture Collection, Northern Regional Research Center (NRRL).

### Systems for Carrying Out the Disclosed Methods

Those skilled in the ail will understand that some or all of the elements of embodiments of the disclosure, and their accompanying operations, may be implemented wholly or partially by one or more computer systems including one or more processors and one or more memory systems. Some elements and functionality may be implemented locally while others may be implemented in a distributed fashion over a network through different servers, e.g., in client-server fashion, for example. In particular, server side operations may be made available to multiple clients in a software as a service fashion.

Those skilled in the art will recognize that, in some embodiments, some of the operations described herein may be performed by human implementation, or through a combination of automated and manual means. When an operation is not fully automated, appropriate components of embodiments of the disclosure may, for example, receive the results of human performance of the operations rather than generate results through its own operation capabilities.

### EXAMPLES

The following examples are given for the purpose of illustrating the methods and tools that may be utilized to predict phenotypic performance of a host cell in industrial culture. These examples are not meant to limit the present disclosure to the embodiments shown. Changes therein and other uses which are encompassed within the spirit of the disclosure, as defined by the scope of the claims, will be recognized by those skilled in the art.

FIG. 1 and FIG. 2 illustrate the general workflow for predicting phenotypic performance of a host cell comprising: 1) small lab-scale culture of a host cell under conditions that would mimic large-scale development in fermenters, 2) obtaining a spent nutrient media sample from small lab-scale culture of the host cell, 3) analyzing the spent nutrient media by mass spectrometry to generate a chemical spectra of the spent nutrient media, 4) providing a predictive model based on a metabolite fingerprint variable and a phenotypic performance variable, and 5) utilizing the predictive model to determine expected phenotypic performance of the host cell by providing the chemical spectra of the first spent media to the predictive model.

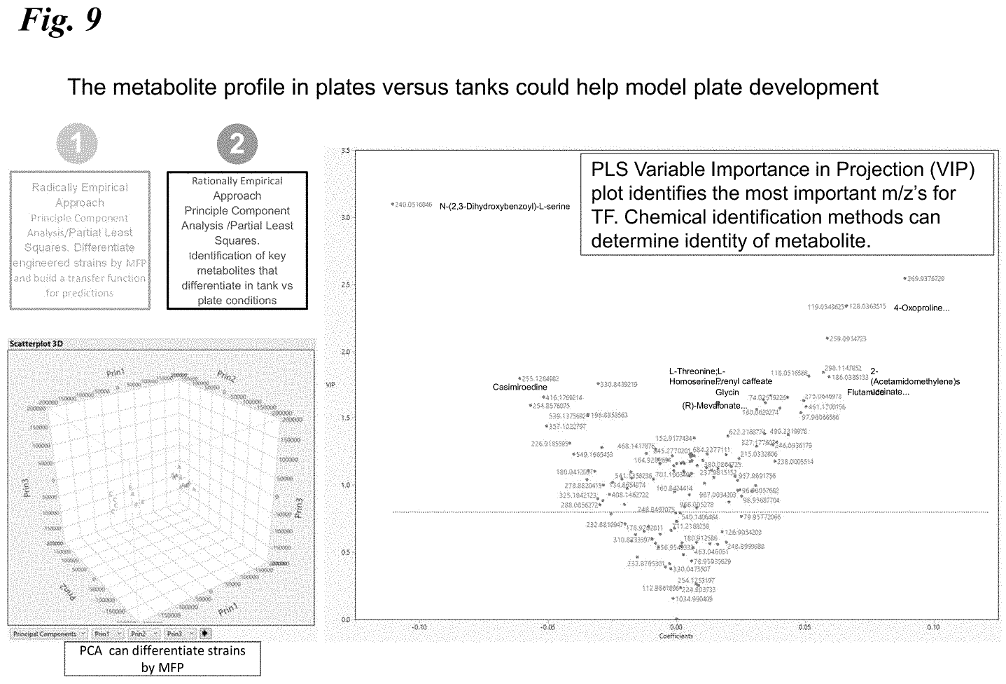

FIG. 9 illustrates other potential applications for metabolite fingerprinting. For example, in some embodiments, metabolite profiles of small lab-scale versus industrial cultures are be used to determine how well the conditions in plates mimic those of large-scale cultures such as bench-top fermenters or bioreactors. Emulating industrial culture conditions could help improve prediction of phenotypic performance.

### Example 1: Generating Transfer Functions with Metabolite Fingerprinting to Predict Phenotypic Performance

This example analyzes multiple parameters used to generate transfer functions for prediction of phenotypic performance of host cells in industrial cultures.

Host cell cultures from a strain improvement program were selected for analysis in small lab-scale and industrial culture (referred to herein as ladder strains or the training dataset). For metabolite fingerprinting, each host cell strain was grown in nutrient media for 48 hours, and the nutrient media was separated from the host cells by centrifugation at 3000 rpm, thereby generating spent nutrient media. The spent nutrient media was then clarified, filtered, and diluted before injection into the ESI-MS using the TOF analyzer. The data obtained by mass spectrometry was converted into the open-source file type .mzML using the msconvert application in ProteoWizard. The MS data was processed by spectral filtering, peak detection, chromatogram building and peak alignment using the MS software MZmine 2. The chemical spectra obtained after processing was referred to as the “metabolite fingerprint” of a host cell, i.e. the metabolite fingerprint variable. Product performance of the host cell in industrial culture was then determined for each strain in the ladder, i.e. the associated phenotypic performance variable. The metabolite fingerprint variable and associated phenotypic performance variable for the training dataset or ladder strains were then analyzed by PLS to generate a predictive model, i.e. transfer function, of phenotypic performance. Further details on generating a predictive model of phenotypic performance are provided in the exemplary workflow described above.

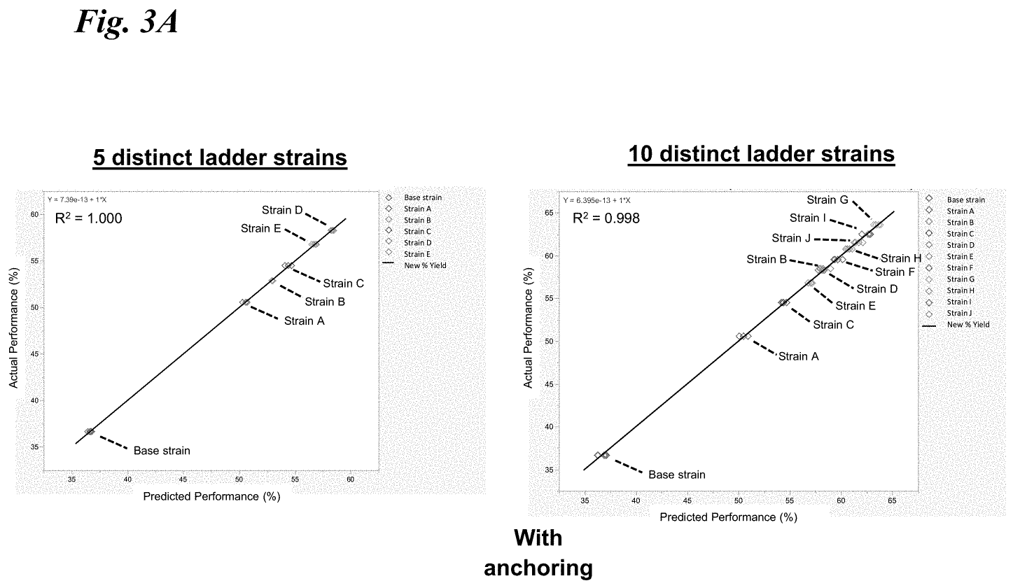

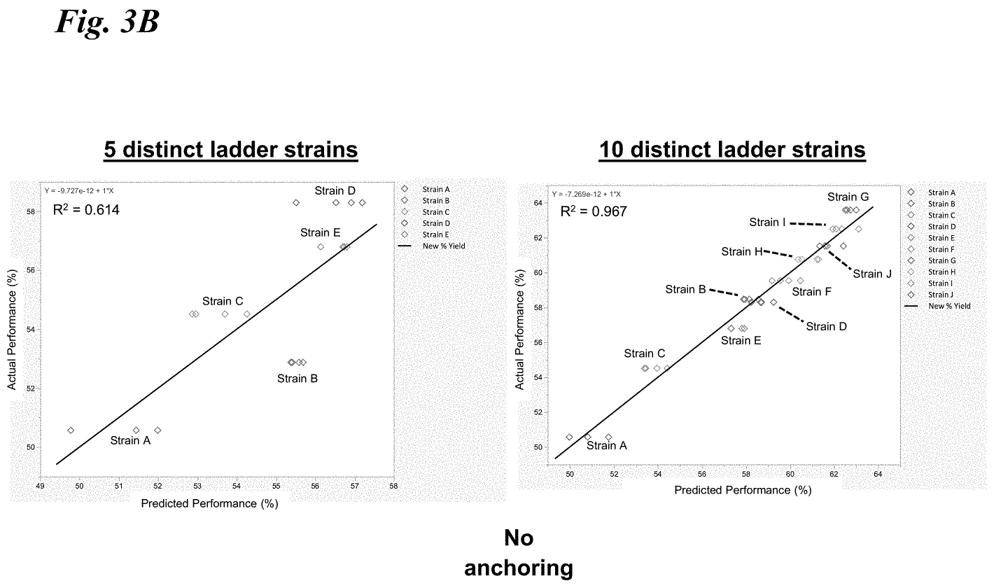

PLS analyses were performed using different ladder strains to determine whether altering number of strains in the ladder influenced transfer function based on metabolite fingerprinting. As shown in FIG. 3A, the transfer function was not significantly affected by using either 5 or 10 ladder strains when an anchor (base) strain was included in the analysis. However, as shown in FIG. 3B, correlation was significantly improved using 10 ladder strains (R² value of 0.967) versus 5 ladder strains (R² value of 0.614) in the absence of an anchor (base) strain.

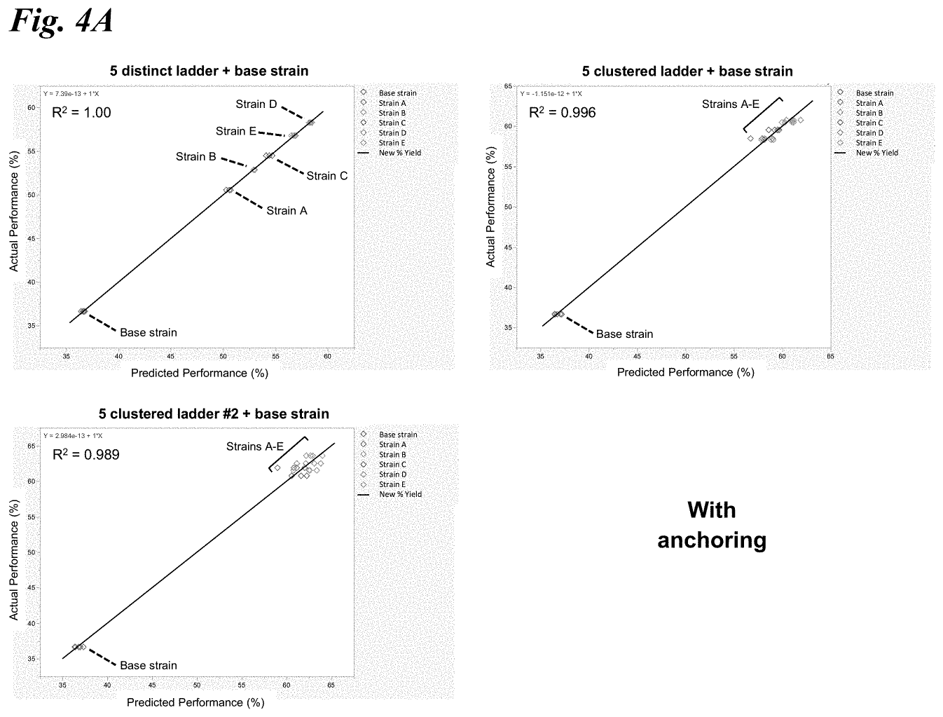

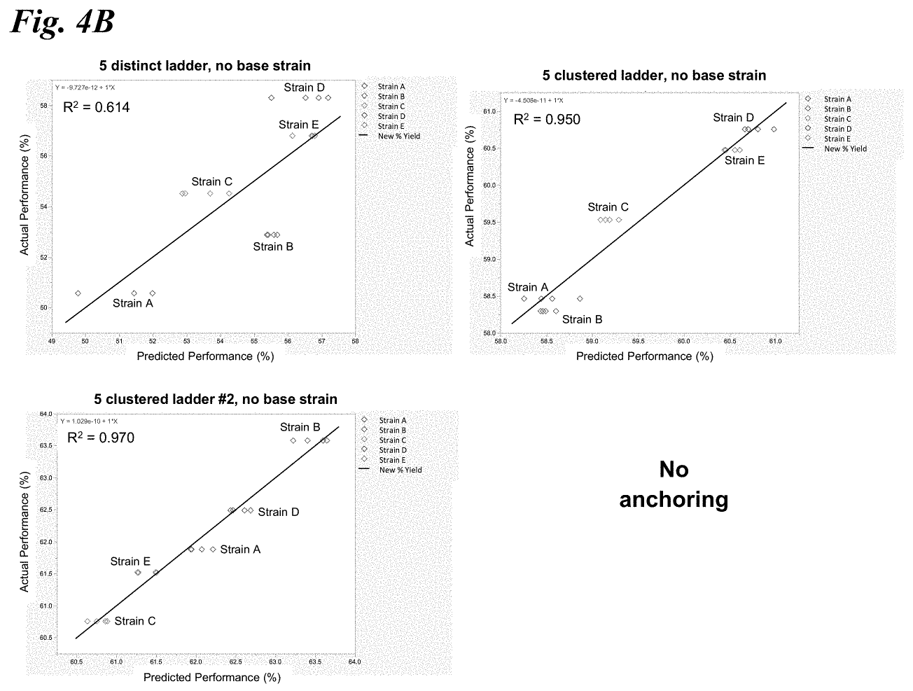

Further experiments were performed to determine whether using clustered or distinct ladder strains influenced transfer function based on metabolite fingerprinting. Clustered ladder strains exhibited similar phenotypic performance in bench-scale industrial culture, while distinct ladder strains exhibited differences in phenotypic performance in bench-scale industrial culture, As shown in FIG. 4A, transfer function was not significantly affected by 5 distinct or clustered ladder strains when an anchor (base) strain was included in the analysis. However, as shown in FIG. 4B, correlation was significantly improved using 5 clustered ladder strains (R² values of 0.950 and 0.970) versus 5 distinct ladder strains (R² value of 0.614) in the absence of an anchor (base) strain.

A summary comparing transfer functions generated from metabolite fingerprinting under various conditions are shown below in Table 3, Low range indicates low range of phenotypic performance while high range indicates high range of phenotypic performance. The R squared and root mean square error (RMSE) values indicate how accurately the model predicts phenotypic performance. RMSE is a measure of deviance of predicted performance from actual performance in industrial cultures.

TABLE 3
Summary of Transfer Function Analyses
Using Metabolite Fingerprinting

| Conditions | TF R² | RMSE |
|---|---|---|
| 5 distinct ladder on the low range with base strain | 0.998 | 3.42 |
| 5 distinct ladder on the low range with no base strain | 0.969 | 4.76 |
| 5 distinct ladder on the high range with base strain | 1 | 3.66 |
| 5 distinct ladder on the high range with no base strain | 0.999 | 3.54 |
| 10 distinct ladder on the high range with base strain | 1 | 2.56 |
| 10 distinct ladder on the high range with no base strain | 0.982 | 3.06 |
| 5 clustered ladder on the kw range with base strain | 0.994 | 2.44 |
| 5 distinct ladder on the low range with no base strain | 0.992 | 4.86 |
| 5 distinct ladder on the high range with base strain | 0.998 | 3.12 |
| 5 distinct ladder on the high range with no base strain | 0.995 | 5.51 |

Overall, these data show that as low as 5 ladder strains can be used to predict phenotypic performance of host cells in bench-scale industrial cultures. Furthermore, the presence of an anchor (base) strain improves correlation using the minimum number of distinct or clustered ladder strains.

### Example 2: Predicting Phenotypic Performance using Traditional Plate-Based Assay Versus Metabolite Fingerprinting

This example compares a traditional plate-based assay and the presently disclosed metabolite fingerprinting transfer function for predicting phenotypic performance of host cells in industrial cultures.

Transfer functions were determined using either a plate-based assay or metabolite fingerprinting of host cells in small lab-scale culture and an associated phenotypic performance measurement. The transfer function for the plate-based assay was generated based on direct product titer measurements of host cells in small lab-scale culture and an associated phenotypic measurement from industrial culture. The transfer function was determined by linear regression. The transfer function for metabolite fingerprinting was generated based on PLS analysis of a metabolite fingerprinting variable and an associated phenotypic performance variable of a host cell, as described in the exemplary workflow of metabolite fingerprinting and Example 1.

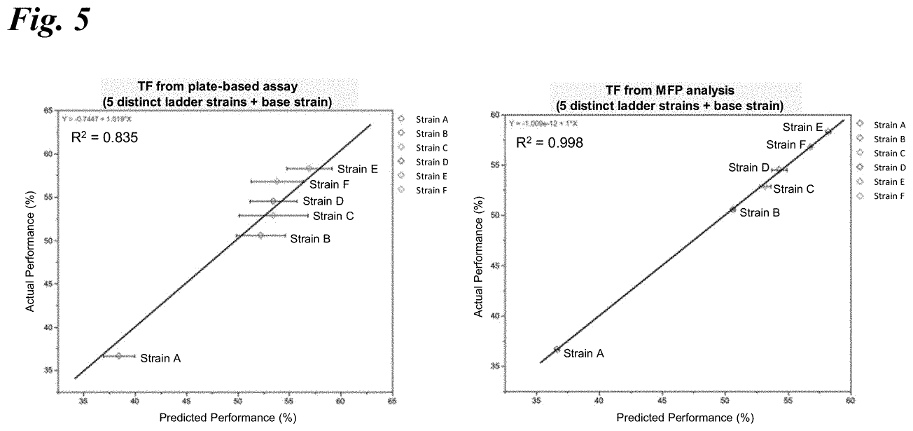

Both transfer functions were established using 5 distinct ladder strains with a low range anchor (base) strain. As shown in FIG. 5 and Table 4, the transfer function generated using metabolite fingerprinting showed a stronger correlation and lower Root Mean Square Error (RMSE) value (R² value of 0.998 and RMSE value of 3.42) than the plate-based assay (R² value of 0.835 and RMSE value of 5.86).

A summary comparing transfer functions generated from metabolite fingerprinting (MFP) and plate-based assays under various conditions are shown below in Table 4. Low range with the base strain indicates low range of phenotypic performance while high range with the base strain indicates high range of phenotypic performance. The R squared and RMSE values indicate how accurately the model predicts phenotypic performance. RMSE is a measure of deviance of predicted performance from actual performance in industrial cultures.

TABLE 4
Transfer Functions from MFP and Plate-Based Assays

| | MFP analysis | | Plate-based assay | |
|---|---|---|---|---|
| Conditions | TF R² | RMSE | TF R² | RMSE |
| 5 distinct ladder on the low range | 0.998 | 3.42 | 0.835 | 5.86 |
| with base strain | | | | |
| 5 distinct ladder on the low range | 0.969 | 4.76 | 0.194 | 5.53 |
| with no base strain | | | | |
| 5 distinct ladder on the high range | 1 | 3.66 | 0.860 | 5.78 |
| with base strain | | | | |
| 5 distinct ladder on the high range | 0.999 | 3.54 | 0.393 | 5.01 |
| with no base strain | | | | |
| 10 distinct ladder on the high range | 1 | 2.56 | 0.393 | 5.01 |
| with base strain | | | | |
| 10 distinct ladder on the high range | 0.982 | 3.06 | 0.393 | 5.01 |
| with no base strain | | | | |
| 5 clustered ladder on the low range | 0.994 | 2.44 | 0.864 | 5.63 |
| with base strain | | | | |
| 5 distinct ladder on the low range | 0.992 | 4.86 | 0.035 | 3.59 |
| with no base strain | | | | |
| 5 distinct ladder on the high range | 0.998 | 3.12 | 0.92 | 5.94 |
| with base strain | | | | |
| 5 distinct ladder on the high range | 0.995 | 5.51 | 0.02 | 3.61 |
| with no base strain | | | | |

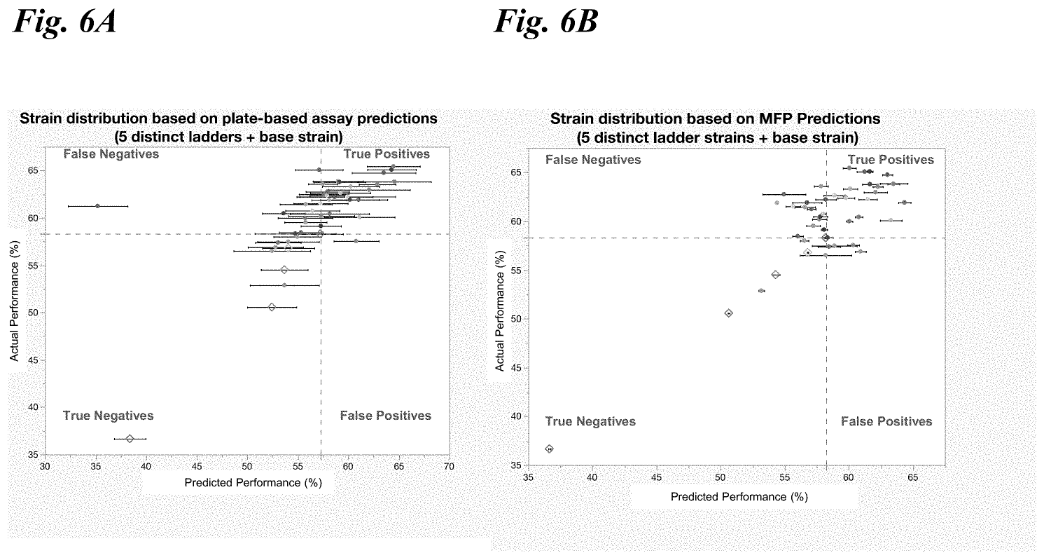

The transfer functions established using 5 distinct ladder strains with a low range anchor (base) strain were then used to predict strain performance of independent or “unknown” strains. The “unknown” strains were plotted based on predicted yield versus actual yield in industrial cultures, FIG. 6A and FIG. 6B show the number of true positives (upper right quadrant), false negatives (upper left quadrant), true negatives (lower left quadrant), and false negatives (lower right quadrant). The reference lines (dashes) were determined based on the highest performing ladder strain in each training set. The results show that although the transfer function generated from the plate-based assay predicted a larger number of true positives, the coefficient of variation (CV) was significantly higher using the plate-based assay compared to the MNP method. The high CV observed using the plate-based assay means a lower signal to noise ratio and could result in the identification of greater number of false positives in industrial culture, which means wasted resources, and increased cost.

Table 5 provides a summary of the statistical comparisons for predicting phenotypic performance using plate-based or metabolite fingerprinting methods under various parameters. The positive predictive value (PPV) and false negative rates (FNR) were determined by the distribution of the “unknown” strains relative to the reference strain. The reference strain was set as the best performing strain in the ladder or training dataset. The actual performance of the reference strain in industrial culture was then used as the reference for determining PPV and FNR. The PPV was calculated by dividing the number of true positive strains by the sum of the number of true positive and false positive strains. The FNR was calculated by dividing the number of false negative strains by the total number of strains screened. The average hit size is the percent improvement over the parent strain. CV is the measure of relative variability, specifically the ratio of standard deviation to the mean. Cross Strain CV indicates CV for the entire dataset, i.e., CV values for the ladder strains and “unknown” strains. CV is calculated on the predicted performance of “unknown” strains. Note that the cross strain CV values for the plate-based assay were the same under various conditions because there was only one performance measurement (i.e., product titer) used to generate a transfer function. As such, regardless of the linear model used, the cross strain CV value does not change for each “unknown” strain.

TABLE 5
Comparison of Methods for Predicting Phenotypic Performance

| | | | | | Avg. | Cross Strain | |
|---|---|---|---|---|---|---|---|
| | PPV | | FNR (%) | | Hit Size | CV | |
| Condition | Plate | MFP | Plate | MFP | (%) | Plate | MFP |
| 5 distinct ladder on die low range | 0.97 | 0.94 | 2.63 | 2.63 | 6.9 | 4.9 | 2.37 |
| with base strain | | | | | | | |
| 5 distinct ladder on the low range | 0.97 | 0.97 | 2.63 | 34.2 | 6.9 | 4.9 | 1.67 |
| with no base strain | | | | | | | |
| 5 distinct ladder on the high range | 0.21 | 0.29 | 0 | 0 | 2.4 | 4.9 | 2.22 |
| with base strain | | | | | | | |
| 5 distinct ladder on the high range | 0.21 | 0.5 | 0 | 2.6 | 2.4 | 4.9 | 1.1 |
| with no base strain | | | | | | | |
| 10 distinct ladder on the high range | 0.21 | 0.4 | 0 | 0 | 2.4 | 4.9 | 1.61 |
| with base strain | | | | | | | |
| 10 distinct ladder on the high range | 0.21 | 1 | 0 | 0 | 2.4 | 4.9 | 1.1 |
| with no base strain | | | | | | | |
| 5 clustered ladder on the low range | 0.96 | 0.89 | 2.63 | 15.3 | 4 | 4.9 | 1.77 |
| with base strain | | | | | | | |
| 5 distinct ladder on the low range | 0.96 | 1 | 2.63 | 48.7 | 4 | 4.9 | 0.32 |
| with no base strain | | | | | | | |
| 5 distinct ladder on the high range | 0.21 | 0.38 | 0 | 0 | 2.4 | 4.9 | 3.15 |
| with base strain | | | | | | | |
| 5 distinct ladder on the high range | 0.21 | 0 | 0 | 10.2 | 2.4 | 4.9 | 0.70 |
| with no base strain | | | | | | | |

Table 5 and FIGS. 6A and 6B shows that the metabolite fingerprinting method had lower average process noise (e.g., 1.3% CV) compared to the plate-based method (4.9% CV). The lower overall noise drives a lower number of replicates required per strain to achieve statistical confidence on strain performance in industrial culture. Thus, metabolite fingerprinting allows for a larger number of strains to be screened in a shorter amount of time (e.g., 1000 strains vs. 700 strains) compared to the plate-based assay. This leads to a larger number of hits using the metabolite fingerprinting strategy compared to the plate-based assay (e.g., 9.5 hits versus 3.4 hits).

### Example 3: Predicting Phenotypic Performance with Metabolite Fingerprinting

This example provides further analysis of predicting phenotypic performance in industrial cultures using a plate-based assay versus metabolite fingerprinting.

Transfer functions were determined using either a plate-based assay or metabolite fingerprinting of host cells in small lab-scale culture and an associated phenotypic performance measurement from an industrial culture. The transfer function for the plate-based assay was generated based on direct product titer measurements in small lab-scale culture and linear regression analysis. The transfer function for metabolite fingerprinting was generated based on PLS analysis of a metabolite fingerprinting variable and an associated phenotypic performance variable, as described in the exemplary workflow of metabolite fingerprinting and Example 1.

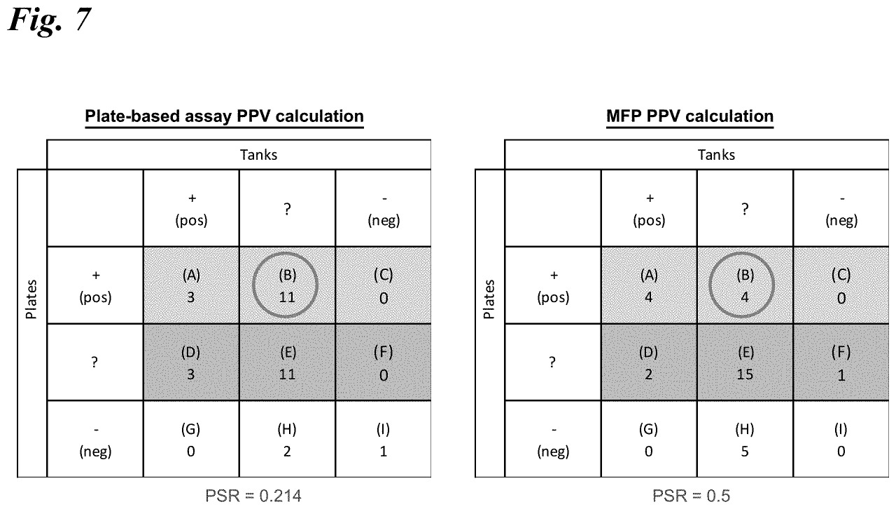

Predictive models were generated based on 35 strains. FIG. 7 shows the accuracy of predicting strain performance using the plate-based (left panel) or metabolite fingerprinting (tight panel) methods. The “A” category signifies a true positive, the “B” category signifies a putative positive, and the “C” category signifies a false positive. Statistical analyses were performed for both the plate-based and metabolite fingerprinting methods to determine where each “unknown” strain falls into categorically and was based on the performance of the “unknown” strain relative to its parental strain. The PPV was calculated by taking the number of strains from the “A” category and dividing it by the sum of strains in the “A”, “B” & “C” categories ([A]/[A+B+C]). These results show that prediction using metabolite fingerprinting reduced the number of strains in the “B” category, resulting in an improved PPV.

The transfer function generated using metabolite fingerprinting was then used to promote strains to large-scale cultures. Strain ID No: 002 was predicted to perform well in industrial culture based on the metabolite profile identified in small lab-scale culture. The actual performance of Strain ID NO: 002 in industrial cultures was unknown and was not previously predicted to perform well using the traditional method.

Table 6 shows the phenotypic performance of the reference strain (Strain ID No: 001) and promoted strain (Strain ID No: 002) in industrial culture. The reference strain was a mid-range, stable performer in culture.

TABLE 6
Phenotypic Performance of Lead Strains in Industrial Culture

| Fermenter | K06 | K05 | K07 | L06 |
|---|---|---|---|---|
| Strain | 001 | 002 | 002 | 002 |
| Yield (w/w %) | 28.6 | 30.8 | 30.4 | 30.8 |
| Productivity (g/L/hr) | 1.4 | 1.5 | 1.4 | 1.5 |
| Final Titer (g/kg) | 64.6 | 69.9 | 68.8 | 71.8 |
| Max OD, 562 nm | 69.6 | 71.2 | 67.0 | 69.6 |
| Final OD, 562 nm | 44.4 | 45.8 | 50.8 | 44.4 |
| Final Tank Weight (g) | 954.00 | 994.30 | 978.10 | 922.70 |
| Feed (g) | 472.5 | 493.9 | 485.6 | 472.4 |
| Status | Good | Good | Good | Good |

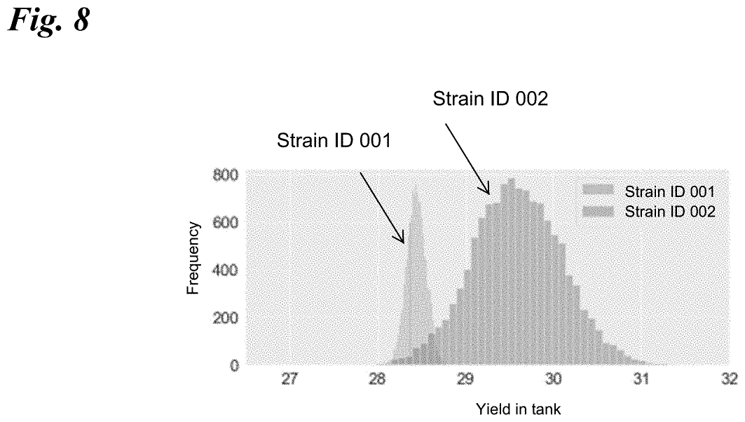

FIG. 8 shows the probability distributions of strain performance for the promoted strain (strain ID No: 002) versus the reference strain (Strain ID No: 001) in industrial culture. Strain ID No: 002 was predicted to perform well in industrial culture based on the model generated by metabolite fingerprinting. These results show that a large distribution of the promoted strain (Strain No: 002) performed better than the reference strain (Strain ID No: 001). Notably, this strain would not have been promoted using traditional methods because it failed to meet the requirements of overall titer and titer/carbon source ratio consumed in small lab-scale culture (Table 6). Thus, metabolite fingerprinting may provide a more accurate prediction of phenotypic performance in industrial culture than traditional plate-based assays.

### Further Embodiments of the Invention

Other subject matter contemplated by the present disclosure is set out in the following numbered embodiments:

1. A method for predicting phenotypic performance of a host cell based on chemical spectra of spent media, said method comprising the steps of:
a) culturing a first host cell in nutrient media and separating the cultured cells from the media, thereby creating a first spent media;
b) analyzing the first spent media via mass spectroscopy to generate a chemical spectra of said first spent media;
c) providing a predictive model of phenotypic performance, said model comprising a metabolite fingerprint variable, and a phenotypic performance variable:
   i) wherein the metabolite fingerprint variable is based on chemical spectra of a plurality of spent media, each spent media having been derived from a host cell culture exhibiting a known phenotypic performance measurement; and
   ii) wherein the phenotypic performance variable is based on the known phenotypic performance measurement associated with each of the chemical spectra of part (i); and
d) utilizing the predictive model to predict the expected phenotypic performance of the first host cell by providing the chemical spectra of the first spent media to the model.

1.1 A computer-implemented method for predicting phenotypic performance of a host cell, said method comprising:
a) providing a first chemical spectra for a first host cell, said first chemical spectra having been produced from an analysis of mass spectroscopy of spent media from a culture of the first host cell;
b) providing a predictive model of phenotypic performance, said model comprising a metabolite fingerprint variable, and a phenotypic performance variable:
   i) wherein the metabolite fingerprint variable is based on chemical spectra of a plurality of spent media, each spent media having been derived from a different host cell culture exhibiting a known phenotypic performance measurement; and
   ii) wherein the phenotypic performance variable is based on the known phenotypic performance measurement associated with each of the chemical spectra of part (i); and
c) utilizing the predictive model to predict the expected phenotypic performance of the first host cell by providing the first chemical spectra to the model.

1.2 A method for predicting phenotypic performance of a host cell based on chemical spectra of spent media, said method comprising the steps of:
a) providing a first spent media from a cultured first host cell;
b) analyzing the first spent media via mass spectroscopy to generate a chemical spectra of said first spent media;
c) providing a predictive model of phenotypic performance, said model comprising a metabolite fingerprint variable, and a phenotypic performance variable:
   i) wherein the metabolite fingerprint variable is based on chemical spectra of a plurality of spent media, each spent media having been derived from a host cell culture exhibiting a known phenotypic performance measurement; and
   ii) wherein the phenotypic performance variable is based on the known phenotypic performance measurement associated with each of the chemical spectra of part (i); and
d) utilizing the predictive model to predict the expected phenotypic performance of the first host cell by providing the chemical spectra of the first spent media to the model.

2. A computer-implemented method for predicting phenotypic performance of a host cell, said method comprising:
a) accessing a training data set comprising a metabolite fingerprint variable, and a phenotypic performance variable;
   i) wherein the metabolite fingerprint variable comprises chemical spectra of a plurality of spent media, each spent media having been derived from a host cell culture exhibiting a known phenotypic performance measurement; and
   ii) wherein the phenotypic performance variable comprises the known phenotypic performance measurements that are associated with each of the spent media of part (i);
b) developing a predictive model that is populated with the training data set; and
c) utilizing the predictive model to predict the phenotypic performance of a first host cell by providing chemical spectra of a first spent media obtained from a culture of the first host cell to the predictive model;
   wherein the chemical spectra of spent media is measured via mass spectroscopy.

3. The method of any one of embodiments 1 to 2, wherein the predictive model is a partial least squares regression of the chemical spectra of spent media and their associated known phenotypic performance measurements.

3.1 A The method of any one of embodiments 1 to 2, wherein the predictive model is selected from the group consisting of partial least squares analysis (PLS), partial least squares discriminant analysis (PLS-DA), orthogonal partial least squares analysis (OPLS), or principal component analysis.

4. The method of any one of embodiments 1 to 3.1, wherein the plurality of spent media in the metabolite fingerprint variable comprises at least 5, 10, 25, 50, 75, 100, 150, 200, or 250 chemical spectra.

5. The method of any one of embodiments 1 to 4, wherein the metabolite fingerprint variable and phenotypic performance variable comprise the chemical spectra and the known phenotypic performance measurements from spent media from host cell cultures that exhibit a range of phenotypic performance measurements.

5.1. The method of embodiment 5, wherein the range of phenotypic performance measurements comprises at least a 10%, 20%, 30%, 40%, 50%, 60%, 70%, 80%, or 90% difference between the lowest and highest known phenotypic performance measurements.

6. The method of embodiment 5, wherein the range of phenotypic performance measurements comprises at least a 2, 3, 4, 5, 6, 7, 8, 9, or 10-fold difference between the lowest and highest known phenotypic performance measurements.

6.1 The method of embodiments 5.1 or 6, wherein the difference between the lowest and highest known phenotypic performance measurements is a relative difference.

6.2 The method of embodiments 5.1 or 6, wherein the difference between the lowest and highest known phenotypic performance measurements is an absolute difference 6.3. The method any one of embodiments 1 to 6.2, wherein the metabolite fingerprint variable comprises a chemical spectra for an anchor strain.

7. The method of any one of embodiments 1 to 6.3, wherein the predicted phenotypic performance is production of a product of interest, said product of interest selected from the group consisting of: a small molecule, enzyme, protein, peptide, amino acid, organic acid, synthetic compound, fuel, alcohol, primary extracellular metabolite, secondary extracellular metabolite, intracellular component molecule, and combinations thereof.

8. The method of any one of embodiments 1 to 7, wherein the model predicts the phenotypic performance of the first host cell in an industrial culture based on the chemical spectra of the first spent media obtained from a small lab-scale culture.

9. The method of any one of embodiments 1 to 8, wherein the metabolite fingerprint variable is based on the chemical spectra of a plurality of spent media from small lab-scale cultures, and wherein the phenotypic performance variable is based on the known phenotypic performance measurements of the host cells in industrial cultures.

10. The method of embodiment 8 or 9, wherein the industrial culture is at least a 0.25, 0.5, 1, 2, 3, 4, 5, 6, 7, 8, 9, 10, 20, 30, 40, or 50 liter culture, and wherein the small lab-scale culture is less than a 1000, 750, 500, 250, 200, 150, 100, 50, or 20 microliter culture.

11. The method of embodiment 10, wherein the small ab-scale culture is a culture from a single well in a 96 or 384-well plate.

12. The method of any one of embodiments 1 to 11, wherein the mass spectroscopy is direct injection electrospray ionization mass spectrometry.

13. The method of any one of embodiments 1 to 12, wherein the mass spectroscopy uses a time-of-flight spectrometer.

14. The method of any one of embodiments 1 to 13, wherein the chemical spectra are based on positive ion mass spectroscopy.

15. The method of any one of embodiments 1 to 13, wherein the chemical spectra are based on negative ion mass spectroscopy.

16 The method of any one of embodiments I to 15, comprising the step of growing the first host cell in an industrial culture in growth media wherein the industrial culture is at least a 0.25, 0.5, 1, 2, 3, 4, 5, 7, 8, 9, 10, 20, 30, 40, or 50 liter culture.

17. The method of embodiment 16 wherein the predicted phenotypic performance is production of a product of interest; and comprising the step of isolating the product of interest from the first host cell industrial culture.

18. A method for predicting phenotypic performance of a host cell based on chemical spectra of spent media, said method comprising the steps of:
a) culturing a first host cell in nutrient media and separating the cultured cells from the media, thereby creating a first spent media;
b) analyzing the first spent media via mass spectroscopy to generate a chemical spectra of said first spent media;
c) providing a predictive model of phenotypic performance, said model comprising a metabolite fingerprint variable, and a phenotypic performance variable:
   i) wherein the metabolite fingerprint variable is based on chemical spectra of a plurality of spent media, each spent media having been derived from a host cell culture exhibiting a known phenotypic performance measurement; and
   ii) wherein the phenotypic performance variable is based on the known phenotypic performance measurement associated with each of the chemical spectra of part (i); and
d) utilizing the predictive model to predict the expected phenotypic performance of the first host cell by providing the chemical spectra of the first spent media to the model; and
e) growing the first host cell in an industrial culture in growth media wherein the industrial culture is at least a 0.25, 0.5, 1, 2, 3, 4, 5, 6, 7, 8, 9, 10, 20, 30, 40, or 50 liter culture;
   wherein first spent media of step (a) and the plurality of spent media of step (c)(i) were all derived from a lab-scale cultures of less than about 5 mL, and wherein the known phenotypic performance measurement of step (c)(ii) was obtained from industrial cultures of at least 0.25 L.

19. A computer-implemented method for predicting phenotypic performance of a host cell, said method comprising:
a) providing a first chemical spectra for a first host cell, said first chemical spectra having been produced from an analysis of mass spectroscopy of spent media from a culture of the first host cell;
b) providing a predictive model of phenotypic performance, said model comprising a metabolite fingerprint variable, and a. phenotypic performance variable:
   i) wherein the metabolite fingerprint variable is based on chemical spectra of a plurality of spent media, each spent media having been derived from a different host cell culture exhibiting a known phenotypic performance measurement; and
   ii) wherein the phenotypic performance variable is based on the known phenotypic performance measurement associated with each of the chemical spectra of part (i); and
c) utilizing the predictive model to predict the expected phenotypic performance of the first host cell by providing the first chemical spectra to the model; and
d) growing the first host cell in an industrial culture in growth media wherein the industrial culture is at least a 0.25, 0.5, 1, 2, 3, 4, 5, 6, 7, 8, 9, 10, 20, 30, 40, or 50 liter culture;
   wherein first spent media of step (a) and the plurality of spent media of step (c)(i) were all derived from a lab-scale cultures of less than about 5 mL, and wherein the known phenotypic performance measurement of step (c)(ii) was obtained from industrial cultures of at least 0.25 L.

20. A method for predicting phenotypic performance of a host cell based on chemical spectra of spent media, said method comprising the steps of:
a) providing a first spent media from a cultured first host cell;
b) analyzing the first spent media via mass spectroscopy to generate a chemical spectra of said first spent media;
c) providing a predictive model of phenotypic performance, said model comprising a metabolite fingerprint variable, and a phenotypic performance variable:
   i) wherein the metabolite fingerprint variable is based on chemical spectra of a plurality of spent media, each spent media having been derived from a host cell culture exhibiting a known phenotypic performance measurement; and
   ii) wherein the phenotypic performance variable is based on the known phenotypic performance measurement associated with each of the chemical spectra of part (i); and
d) utilizing the predictive model to predict the expected phenotypic performance of the first host cell by providing the chemical spectra of the first spent media to the model; and
e) growing the first host cell in an industrial culture in growth media wherein the industrial culture is at least a 0.25, 0.5, 1, 2, 3, 4, 5, 6, 7, 8, 9, 10, 20, 30, 40, or 50 liter culture;
   wherein first spent media of step (a) and the plurality of spent media of step (c)(i) were all derived from a lab-scale cultures of less than about 5 mL, and wherein the known phenotypic performance measurement of step (c)(ii) was obtained from industrial cultures of at least 0.25 L.

21. A method for preparing a predictive model capable of predicting phenotypic performance of a host cell based on chemical spectra of spent media, said method comprising the steps of:
a) providing a plurality of chemical spectra derived from mass spectroscopy analysis of spent media, each spent media having been derived from a lab-scale culture of a host cell exhibiting a known phenotypic performance measurement of the same host cell in an industrial-scale culture; and
b) conducting a partial least squares analysis of the plurality of the chemical spectra of step (a) and their associated known phenotypic performance measurements, thereby generating a predictive model capable of predicting a first host cell's phenotypic performance in industrial scale cultures based on the chemical spectra of spent media from a lab-scale culture of the first host cell.

22. The embodiment of claim 21, wherein the partial least squares analysis is selected from the group consisting of, partial least squares discriminant analysis (PLS-DA), orthogonal partial least squares analysis (OPLS), or principal component analysis.

23. The method of any one of embodiments 21-22, wherein the plurality of chemical spectra comprises at least 5, 10, 25, 50, 75, 100, 150, 200, or 250 chemical spectra.

24. The method of any one of embodiments 21-23 wherein the plurality of chemical spectra comprise chemical spectra derived from a lab-scale culture of a host cell exhibiting a range of phenotypic performance measurements of the same host cells in an industrial-scale cultures.

25. The method of embodiment 24, wherein the range of phenotypic performance measurements comprises at least a 2, 3, 4, 5. 6, 7, 8, 9, or 10-fold difference between the lowest and highest known phenotypic performance measurements.

25.1. The method of embodiment 24, wherein the range of phenotypic performance measurements comprises at least a 10%, 20%, 30%, 40%, 50%, 60%, 70%, 80%, or 90% difference between the lowest and highest known phenotypic performance measurements.

25.2 The method of embodiments 25 or 25., wherein the difference between the lowest and highest known phenotypic performance measurements is a relative difference.

25.3 The method of embodiments 25 or 25.1, wherein the difference between the lowest and highest known phenotypic performance measurements is an absolute difference.

26.The method any one of embodiments 21 to 25.3, wherein the plurality of chemical spectra comprises a chemical spectra for an anchor strain.

27. The method of any one of embodiments 24 to 26, wherein the predicted phenotypic performance is production of a product of interest, said product of interest selected from the group consisting of: a small molecule, enzyme, protein, peptide, amino acid, organic acid, synthetic compound, fuel, alcohol, primary extracellular metabolite, secondary extracellular metabolite, intracellular component molecule, and combinations thereof.

28. A method for selecting a host cell for industrial culture comprising the steps of:
a) providing a plurality of test chemical spectra produced from mass spectroscopy analysis of spent media from lab-scale cultures of a plurality of test host cells;
b) providing a predictive model of phenotypic performance, said model comprising a metabolite fingerprint variable, and a phenotypic performance variable:
   i) wherein the metabolite fingerprint variable comprises a ladder of chemical spectra, said ladder of chemical spectra having been produced from mass spectroscopy analysis of spent media from lab-scale cultures of a plurality of host cells exhibiting a range of known phenotypic performance measurements in industrial culture; and
   ii) wherein the phenotypic performance variable comprises the known phenotypic performance measurement in industrial cultures associated with each the of chemical spectra of the ladder of chemical spectra of part (i); and
c) utilizing the predictive model to predict the expected phenotypic performance of the test host cells in industrial culture by providing the test chemical spectra to the model; and
d) selecting a test host cell for culture based, in part, on the predicted phenotypic performance of the test host cells in industrial culture.

29. The method of embodiment 28, comprising:
e) growing the test host cell selected in step (d) in an industrial culture.

30. The method of embodiments 28 or 29, wherein the predictive model is a partial least squares regression of the ladder of chemical spectra of spent media and their associated known phenotypic performance measurements.

31. The method of any one of embodiments 28 to 30, wherein the predictive model is selected from the group consisting of partial least squares analysis (PLS), partial least squares discriminant analysis (PLS-DA), orthogonal partial least squares analysis (OPLS), or principal component analysis.

32. The method of any one of embodiments 28 to 31, wherein the metabolite fingerprint variable comprises at least 5, 10, 25, 50, 75, 100, 150, 200, or 250 chemical spectra.

33. The method of any one of embodiments 28-32, wherein the range of known phenotypic performance measurements comprises at least a 2, 3, 4, 5, 6, 7, 8, 9, or 10-fold difference between lowest and highest known phenotypic performance measurements.

33.1. The method of any one of embodiments 28-32, wherein the range of phenotypic performance measurements comprises at least a 10%, 20%, 30%, 40%, 50%, 60%, 70%, 80%, or 90% difference between the lowest and highest known phenotypic performance measurements.

33.2 The method of embodiments 33 or 33.1, wherein the difference between the lowest and highest known phenotypic performance measurements is a relative difference.

33.3 The method of embodiments 33 or 33.1, wherein the difference between the lowest and highest known phenotypic performance measurements is an absolute difference.

34. The method any one of embodiments 28-33.3, wherein the metabolite fingerprint variable comprises a chemical spectra for an anchor strain.

35. The method of any one of embodiments 28 to 34, wherein the predicted phenotypic performance is production of a product of interest, said product of interest selected from the group consisting of: a small molecule, enzyme, protein, peptide, amino acid, organic acid, synthetic compound, fuel, alcohol, primary extracellular metabolite, secondary extracellular metabolite, intracellular component molecule, and combinations thereof.

36. The method of any one of embodiments 28 to 35, wherein the industrial cultures are at least a 0.25, 0.5. 1, 2, 3, 4, 5, 6, 7, 8, 9, 10, 20, 30, 40, or 50 liter cultures, and wherein the small lab-scale cultures are less than a 1000, 750, 500, 250, 200, 11.50, 100, 50, or 20 microliter cultures.

37. The method of any one of embodiments 28 to 36, wherein each of the small lab-scale cultures of the plurality of test host cells is from a single well in a 96 or 384-well plate.

38. The method of any one of embodiments 28 to 37, wherein the mass spectroscopy is direct injection electrospray ionization mass spectrometry.

39. The method of any one of embodiments 28 to 38, wherein the mass spectroscopy uses a time-of-flight spectrometer.

40. The method of any one of embodiments 28 to 39, wherein the chemical spectra are based on positive ion mass spectroscopy.

41. The method of any one of embodiments 28 to 39, wherein the chemical spectra are based on negative ion mass spectroscopy.

42. The method of any one of embodiment 29-41 wherein the predicted phenotypic performance is production of a product of interest; and comprising:
f) isolating the product of interest from the test host cell industrial culture of step (e).

43. A method for generating a metabolite fingerprint, said method comprising the steps of:
a) obtaining a spent nutrient media sample from host cells in small-culture;
b) analyzing the spent nutrient media sample by mass spectrometry; and
c) processing the mass spectrometry data by spectral filtering, mass detection, chromatogram building, and peak alignment.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. it is intended that the following Claims define the scope of the disclosure and that methods and structures within the scope of these claims and their equivalents be covered thereby.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as, an acknowledgment or any form of suggestion that they constitute valid prior art or form part of the common general knowledge in any country in the world.

1. A computer-implemented method for predicting phenotypic performance of a host cell, said method comprising:
a) providing a first chemical spectra for a first host cell, said first chemical spectra having been produced from an analysis of mass spectroscopy of a first spent media from a culture of the first host cell;
b) providing a predictive model of phenotypic performance, said model comprising a metabolite fingerprint variable, and a phenotypic performance variable:
i) wherein the metabolite fingerprint variable is based on chemical spectra of a plurality of spent media, each spent media having been derived from a plurality of different host cells and
ii) wherein the phenotypic performance variable is based on known phenotypic performance measurements associated with each of the plurality of different host cells of part (i); and
c) utilizing the predictive model to predict the expected phenotypic performance of the first host cell by providing the first chemical spectra to the model.

2. The method of claim 1, wherein the predictive model is a partial least squares regression of the chemical spectra of the plurality of spent media and their associated known phenotypic performance measurements.

3. The method of claim 1, wherein the predictive model is selected from the group consisting of partial least squares analysis (PLS), partial least squares discriminant analysis (PLS-DA), orthogonal partial least squares analysis (OPLS), or principal component analysis.

4. The method of claim 1, wherein the metabolite fingerprint variable comprises at least 5, 10, 25, 50, 75, 100, 150, 200, or 250 chemical spectra.

5. The method of claim 1, wherein the metabolite fingerprint variable and phenotypic performance variable comprise the chemical spectra and the known phenotypic performance measurements from spent media from host cell cultures that exhibit a range of phenotypic performance measurements, wherein the range of phenotypic performance measurements comprises at least a 10%, 20%, 30%, 40%, :50%, 60%, 70%, 80%, or 90% relative difference between the lowest and highest known phenotypic performance measurements.

6. The method of claim 1, wherein the metabolite fingerprint variable and phenotypic performance variable comprise the chemical spectra and the known phenotypic performance measurements from spent media from host cell cultures that exhibit a range of phenotypic performance measurements, wherein the range of phenotypic performance measurements comprises at least a 2, 3, 4, 5, 6, 7, 8, 9, or 10-relative fold difference between the lowest and highest known phenotypic performance measurements.

7. The method of claim 1, wherein the predicted phenotypic performance is production of a product of interest, said product of interest selected from the group consisting of: a small molecule, enzyme, protein, peptide, amino acid, organic acid, synthetic compound, fuel, alcohol, primary extracellular metabolite, secondary extracellular metabolite, intracellular component molecule, and combinations thereof.

8. The method of claim 1, wherein the metabolite fingerprint variable is based on the chemical spectra of a plurality of spent media from small lab-scale cultures, and wherein the phenotypic performance variable is based on the known phenotypic performance measurements of the plurality of different host cells in industrial cultures.

9. The method of claim 8, wherein the industrial cultures are at least 3 liter cultures, and wherein the small lab-scale cultures are less than 1000 microliter cultures.

10. The method of claim 1, wherein the mass spectroscopy is direct injection electrospray ionization mass spectrometry.

11. The method of claim 1, wherein the mass spectroscopy uses a time-of-flight spectrometer.

12. The method of claim 1, wherein the chemical spectra are based on positive ion mass spectroscopy.

13. The method of claim 1. wherein the chemical spectra are based on negative ion mass spectroscopy,

14. A computer-implemented method for predicting phenotypic performance of a host cell, said method comprising:
a) providing a first chemical spectra for a first host cell, said first chemical spectra having been produced from an analysis of mass spectroscopy of a first spent media from a culture of the first host cell;
b) providing a predictive model of phenotypic performance, said model comprising a metabolite fingerprint variable, and a phenotypic performance variable:
i) wherein the metabolite fingerprint variable is based on chemical spectra of a plurality of spent media, each spent media having been derived from a plurality of different host cells; and
ii) wherein the phenotypic performance variable is based on known phenotypic performance measurements associated with each of the plurality of different host cells of part (i); and
c) utilizing the predictive model to predict the expected phenotypic performance of the first host cell by providing the first chemical spectra to the model; and
d) growing the first host cell in an industrial culture in growth media wherein the industrial culture is at least a 02 liter culture;
wherein first spent media of step (a) and the plurality of spent media of step (c)(i) were all derived from a lab-scale cultures of less than about 5 mL, and wherein the known phenotypic performance measurements of step (c)(ii) were obtained from industrial cultures of at least 0.25 L.

15. The claim of claim 14, wherein the partial least squares analysis is selected from the group consisting of, partial least squares discriminant analysis (PLS-DA), orthogonal partial least squares analysis (OPLS), or principal component analysis.

16. The method of claim 14, wherein the plurality of chemical spectra comprises at least 5, 10, 25, 50, 75, 100, 150, 200, or 250 chemical spectra.

17. The method of claim 14 wherein the plurality of chemical spectra comprise chemical spectra from host cells exhibiting a range of phenotypic performance measurements in industrial-scale cultures, wherein the range of phenotypic performance measurements comprises at least a 2, 3, 4, 5, 6, 7, 8, 9, or 10-fold relative difference between the lowest and highest known phenotypic performance measurements.

18. The method of claim 14 wherein the plurality of chemical spectra comprise chemical spectra from host cells exhibiting a range of phenotypic performance measurements in industrial-scale cultures, wherein the range of phenotypic performance measurements comprises at least a 10%, 20%, 30%, 40%, 50%, 60%, 70%, 80%, or 90% relative difference between the lowest and highest known phenotypic performance measurements.

19. The method of claim 14, wherein the predicted phenotypic performance is production of a product of interest, said product of interest selected from the group consisting of: a small molecule, enzyme, protein, peptide, amino acid, organic acid, synthetic compound, fuel, alcohol, primary extracellular metabolite, secondary extracellular metabolite, intracellular component molecule, and combinations thereof.

20. A method for selecting a host cell for industrial culture comprising the steps of:
a) providing a plurality of test chemical spectra produced from mass spectroscopy analysis of spent media from lab-scale cultures of a plurality of test host cells;
b) providing a predictive model of phenotypic performance, said model comprising a metabolite fingerprint variable, and a phenotypic performance variable:
i) wherein the metabolite fingerprint variable comprises a ladder of chemical spectra, said ladder of chemical spectra having been produced from mass spectroscopy analysis of spent media from small lab-scale cultures of a plurality of host cells exhibiting a range of known phenotypic performance measurements in industrial culture; and.
ii) wherein the phenotypic performance variable comprises the known phenotypic performance measurement in industrial cultures associated with each the of chemical spectra of the ladder of chemical spectra of part (i); and
c) utilizing the predictive model to predict the expected phenotypic performance of the test host cells in industrial culture by providing the test chemical spectra to the model; and
d) selecting a test host cell for culture based, in part, on the predicted phenotypic performance of the test host cells in industrial culture.
